(19) United States
(12) Patent Application Publication
(10) Pub. No.: US 2004/0030103 A1
(43) Pub. Date: Feb. 12, 2004

(54) Anti-idiotype inducing antibodies, comprising at least one epitope from angiodenin, for the inhibition of angiogenesis

(76) Inventors: Ronald Burch, Wilton, CT (US); Daniel A Soltis, Belle Mead, NJ (US); Jenny Z Zhang, Cranbury, NJ (US)

Correspondence Address: Darby & Darby, PO Box 5257, New York, NY, 10150-5257, US

(21) Appl. No.: 10/240,953

(22) PCT Filed: Mar. 28, 2001
(86) PCT No.: PCT/US01/10115

(51) Int. Cl. C07K 16/44

(52) U.S. Cl. 530/387.2; 530/387.3

(57) ABSTRACT

Variants of an immunoglobulin variable domain, said immunoglobulin variable domain comprising (A) at least one CDR region and (B) framework regions flanking said CDR, said variant comprising: (a) said CDR region havind added or substituted therein at least one binding sequence and (b) said flanking framework regions, wherein said binding sequence is heterologous to said CDR and is a binding sequence from a binding site of a binding pair, and wherein said binding sequence is a receptor-binding portion of angiogenin. Additionally, amino acid residues in the flanking and/or non-flanking framework regions may have been deleted, substituted or inserted. Also claimed are molecules comprising said variants. Said molecules may be immunoglobulins. Nucleic acids encoding said variants, and cells and recombinant non-human hosts containing said nucleic acids. Pharmaceutical compositions comprising an effective amount of said variants. Vaccines comprising an effective amount of said variants, especially to induce an anti-idiotype response. Method of treating or preventing a disease using an effective amount of said variants or of the nucleic acids encoding said variants.

### CROSS-REFERENCE TO RELATED APLICATION

This application claims priority under 35 U.S.C. § 119 based upon U.S. Provisional Application No. 60/194,729, the entire disclosure of which is incorporated herein by reference.

### FIELD OF THE INVENTION

The present invention relates to inhibition of angiogenesis. In particular, the invention provides synthetic constructs that can provide an antibody-based approach to inhibiting the activity of angiogenin. The synthetic constructs are useful in the treatment of cancer and other diseases associated with inappropriate vascularization.

### BACKGROUND OF THE INVENTION

Cancer remains the second leading cause of death in the United States. There were an estimated 563,100 cancer deaths in 1999. Each year, about 1,222,000 new cancer cases are diagnosed. Among them, solid tumors that depend upon angiogenesis for growth, such as lung, breast, prostate, and colorectal cancers, are most common.

Non-surgical therapy for breast, lung, colon, and ovarian cancers, as well as many other solid tumors, is presently poor. While initial therapies for breast and ovarian cancer with taxanes result in some response by most patients, nearly all patients with ovarian cancer and some patients with breast cancer relapse. Presently, no useful non-surgical therapies of any kind exist for late stage colorectal or lung cancer.

Angiogenesis plays a critical role in cancer development. Tumor cells often have the ability to produce angiogenic factors (including angiogenin) that stimulate new blood vessel growth to support their increasing demand for oxygen and nutrients to sustain rapid growth. When tumors are implanted into isolated perfused organs, they fail to grow beyond a few millimeters in diameter. However, when implanted into mice, the same tumors grow rapidly and kill their hosts. The reason for these differences is that the tumors, when implanted into animals become highly vascularized, while in the organs, capillary formation can not be supported (Gimbrone, et al., Nature 1969, 222: 33). This led to the hypothesis that tumors are angiogenesis-dependent and resulted in the isolation and identification of several angiogenic factors secreted by tumor and stromal cells that support their growth (Folkman and Kiagsburn, Science, 1987, 235: 442).

In addition to supporting tumor growth, angiogenesis is important in other diseases. Uncontrolled angiogenesis contributes to the progression of rheumatoid arthritis, diabetic retinopathy, endometriosis, and psoriasis. Exuberant growth of blood vessels results in the formation of hemangiomas and arteriovenous malformations that cause a variety of clinical problems ranging from cosmetic complications to life-threatening hemorrhages.

Several angiogenin-derived agents have demonstrated anti-angiogenic activity. A monoclonal antibody to human angiogenin has been shown to suppress tumor growth in athymic mice (Olson et al, Cancer Res, 1994, 54:4576). Peptides complementary to the receptor-binding site of angiogenin have been shown to block angiogenin binding to actin and inhibit angiogenesis (Gho et al, J Biol Chem, 1997, 272:24294). Antibodies to actin also showed anti-angiogenic activity (Hu et al, Proc. Natl. Acad. Sci. USA, 1993, 90:1217).

### Angiogenin

Angiogenin is a 14 kDa, non-glycosylated polypeptide so named for its ability to induce new blood vessel growth (Fett et al., Biochemistry, 1985, 24:5480). It is produced by several growing cell types including vascular endothelial cells, aortic smooth muscle cells, fibroblasts (from embryos, new-borns and adults), and some tumors such as colon carcinomas, ovarian carcinomas, and breast cancers (Moenner el al., Eur. J. Biochem. 1994. 226:483). It is homologous to pancreatic ribonuclease (Strvdom et al., Biochem. 1985. 24:5486) although it has distinct ribonucleolytic activity (Shapiro et al., Biochem.; 1986, 25:3527-3532). Angiogenin is a member of the RISBASE (ribonucleases with special biological actions) family of ribonucleases (D'Alessio et al. Trends Biochem. Sci., 1991. 16:104). However, it has not been established what role the ribonucleolytic activity of angiogenin plays in angiogenesis induced by this protein.

Angiogenin was originally isolated from the culture supernatant of a human adenocarcinoma cell line. HT-29,, based on its ability to initiate vascularization in a chicken embryo membrane (Fedd, et al., Biochem., 1985, 24: 5480). It induces angiogenesis in other models as well, e.g., the rabbit comea (Denefle, et al. Gene 1987, 56:61-70). In normal human plasma, angiogenin is present at a concentration of 60-120 ng/ml. Elevated concentrations of angiogenin in cancer patients were found to correlate with more advanced invasiveness and metastasis of the tumors.

The cellular receptor for angiogenin appears to be an actin (Hu, et al, Proc. Natl. Acad. Sci. USA 1983), to which it binds with high affinity. The receptor-binding domain of angiogenin has been localized to amino acids 58-70 (Hallahan, et al., Proc. Natl. Acad. Sci. USA 1991, 88:2222-2236). Both actin and anti-actin antibodies have been shown to inhibit angiogenin-induced angiogenesis, suggesting that binding of angiogenin to actin is essential for mediating angiogenesis (Hu, et al., supra). Angiogenin binding to endothelial cells initiates a variety of activities suggestive of signal transduction following ligand-receptor interaction, including transient activation of phosphatidylinositol-specific phospholipase C and diacyclglycerol lipase, release of arachidonate and generation of prostaglandins (Bicknell and Vallee, Proc. Natl. Acad. Sci. USA 1989, 86:1573-1577; Moore and Riordan, Biochemistry 1990, 29: 228-233). Activation of endothelial cells by angiogenin induces them to proliferate, thereby promoting new vessel formation (Bond, et al., Biochim. Biophys. Acta 1993, 1162: 177. Hu, et al, Proc. Natl. Acad. Sci. USA 1993, 90: 1217; Folkman and Klagsburn, Science 1987, 235: 442).

In addition to activating endothelial cells upon binding, once angiogenin becomes bound to actin, at least some of the angiogenin-actin complex is released from the cell surface (Hu and Riordan. Biochem. Biophys. Res. Commun. 1993, 197:682-687).

Angiogenin-actin complex released upon binding accelerates the generation of plasmin that degrades proteins in the extracellular matrix, facilitating endothelial cell invasion and migration through the extracellular matrix, an essential step in the initiation of angiogenesis (Hu and Riordan, Biochem. Biophys. Res. Commun. 1993, 197:682).

### Immunoglobulins and Immune Response

The basic unit of antibody immunoglobulin structure is a complex of four polypeptides—two identical low molecular weight or “light” chains and two identical high molecular weight or “heavy” chains—linked together by both non covalent associations and by disulfide bonds. Each light and heavy chain of an antibody has a variable region at its amino terminus and a constant domain at its carboxyl terminus. The variable regions are distinct for each antibody and contain the antigen binding site. Each variable domain is comprised of four relatively conserved framework regions and three regions of sequence hypervariability termed complementarity determining regions or “CDRs”. For the most part, it is the CDRs that form the antigen binding site and confer antigen specificity. The constant domains are more highly conserved than the variable regions, with slight variations due to haplotypic differences.

Based on their amino acid sequences, light chains are classified as either kappa or lambda. The constant region of heavy chains is composed of multiple domains (CH1, CH2, CH3 . . . CHx), the number depending upon the particular antibody class. The CH1 region is separated from the CH2 region by a hinge region that allows flexibility in the antibody. The variable region of each light chain aligns with the variable region of each heavy chain, and the constant region of each light chain aligns with the first constant region of each heavy chain. The CH2-CHx domains of the constant region of a heavy chain form an “Fc region” that is responsible for the effector functions of the immunoglobulin molecule, such as complement binding and binding to the Fc receptors expressed by lymphocytes, granulocytes, monocyte lineage cells, killer cells, mast cells, and other immune effector cells.

In modern medicine, immunotherapy or vaccination has virtually eradicated diseases such as polio, tetanus, tuberculosis, chicken pox, measles, hepatitis, etc. The approach using vaccinations has exploited the ability of the immune system to prevent infectious diseases.

There are two types of immunotherapy, active immunotherapy and passive immunotherapy. In active immunotherapy, an antigen is administered in a vaccine to a patient, so as to elicit a long-lasting protective immune response against the antigen. Passive immunotherapy involves the administration of protective antibodies to a patient to elicit an acute immune response that lasts only as long as the antibody is present. Antibody therapy is conventionally characterized as passive since the patient is not the source of the antibodies. However, the term passive may be misleading because a patient may produce anti-idiotype secondary antibodies which in turn provoke an immune response that is cross-reactive with the original antigen. Immunotherapy where the patient generates anti-idiotypic antibodies is often more therapeutically effective than passive immunotherapy because the patient's own immune system generates an active immune response against cells bearing the particular antigen well after the initial infusion of protective antibody has cleared from the system.

In an anti-idiotypic response, antibodies produced initially during an immune response or introduced into an organism will carry unique new epitopes to which the organism is not tolerant, and therefore will elicit production of secondary antibodies (termed “Ab2”), some of which are directed against the idiotype (i.e., the antigen binding site) of the primary antibody (termed “Ab1”), i.e., the antibody that was initially produced or introduced exogenously. These secondary antibodies or Ab2, likewise will have an idiotype that will induce production of tertiary antibodies (termed “Ab3”), some of which will recognize the antigen binding site of Ab2, and so forth. This is known as the “network” theory. Some of the secondary antibodies will have a binding site that is an analog of the original antigen, and thus will reproduce the “internal image” of the original antigen. Tertiary or Ab3 antibodies that recognize this antigen binding site of the Ab2 antibody will also recognize the original antigen.

Therefore, anti-idiotypic antibodies have binding sites that are similar in conformation and charge to the antigen, and can elicit the same or a greater response than that of the target antigen itself. Administration of an exogenous antibody that can elicit a strong anti-idiotypic response can thus serve as an effective vaccine, by eliciting a self-propagating anti-idiotype immune response.

Clinically useful anti-idiotypic responses are rare when intact antibodies are used as the immunogen. The ability to deliver antibodies that reproducibly cause the generation of such an anti-idiotypic response is difficult (Foon, et al. J. Clin. Invest. 1995, 9:334-342; Madiyalakan, et al., Hybridoma 1995, 14: 199-203). One of the reasons for the failure generally to generate an anti-idiotypic response is that Ab1, while exogenous, is still very similar to self as all antibodies have very similar structure and anti-idiotypic responses to self molecules tend to be very limited. A strong anti-idiotypic cascade has been observed when Ab1 has been structurally damaged (Madiyalakan et al., Hybridoma, 1995, 14:199-203) rendering the antibody more foreign. U.S. Pat. No. 4,918,164 discloses direct administration to the subject of exogenously produced anti-idiotype antibodies that are raised against the idiotype of an anti-tumor antibody. After administration, the subject produces anti-antibodies that not only recognize these anti-idiotype antibodies, but also recognize the original tumor epitope, thereby directing complement activation and other immune system responses to a foreign entity to attack the tumor cell that expresses the tumor epitope.

PCT Publication WO 99/25378 relates to molecules, particularly antibodies, that bind one member of a binding pair and have at least one complementarity determining region (CDR) that contains the amino acid sequence of a binding site for that member of the binding pair. The binding site is derived from the other member of the binding pair. It also relates to methods for treating, diagnosing, or screening for diseases and disorders associated with the expression of the member of the binding pair using the modified antibodies.

PCT Publication WO 99/25379 relates to vaccine compositions of antibodies in which one or more variable region cysteine residues that form intrachain disulfide bonds have been replaced with amino acid residues that do not contain a sulfhydryl group and, therefore, do not form disulfide bonds. It also relates to use of the vaccine compositions to treat or prevent certain diseases and disorders.

In sum, there is a need in the art to identify effective inhibitors of angiogenesis. There is a further need in the art to inhibit pathogenic angiogenesis associated with cancer and the other conditions mentioned above. The present invention addresses these and other needs in the art with the discovery of an effective antibody-based angiogenin antagonist system.

### SUMMARY OF THE INVENTION

The present invention provides variants of an immunoglobulin variable domain. The immunoglobulin variable domain comprises (A) at least one CDR region and (B) framework regions flanking said CDR. The variant comprises (a) the CDR region having added or substituted therein at least one binding sequence and (b) the flanking framework regions, wherein the binding sequence is heterologous to the CDR and is a binding sequence from a binding site of a binding pair, and wherein said binding sequence is a receptor-binding portion of angiogenin.

In further embodiment, (i) one or more amino acid residues in one or more of the flanking framework regions has been substituted or deleted, (ii) one or more amino acid residues has been added in one or more of the flanking framework regions, or (iii) a combination of (i) and (ii). Alternatively, (i) one or more amino acid residues in one or more framework regions other than the framework regions flanking the CDR has been substituted or deleted, (ii) one or more amino acid residues has been added in one or more framework regions other than the framework regions flanking said CDR, or (iii) a combination of (i) and (ii). In yet another alternative, (i) one or more amino acid residues in one or more of the flanking framework regions has been substituted or deleted, (ii) one or more amino acid residues has been added in one or more of the flanking framework regions, or (iii) a combination of (i) and (ii); and (iv) one or more amino acid residues in one or more framework regions other than the framework regions flanking the CDR has been substituted or deleted, (v) one or more amino acid residues has been added in one or more framework regions other than the framework regions flanking said CDR, or (vi) a combination of (iv) and (v).

The present invention also provide variants in which the CDR region has added or substituted therein at least one amino acid sequence which is heterologous to the CDR and the flanking framework regions, wherein the heterologous sequence is capable of binding to a target sequence or molecule, and wherein the heterologous sequence is a receptor-binding portion of angiogenin. Again, (i) one or more amino acid residues in one or more of the flanking framework regions may be substituted or deleted, (ii) one or more amino acid residues may be added in one or more of the flanking framework regions, or (iii) a combination of (i) and (ii); (i) one or more amino acid residues in one or more framework regions other than the framework regions flanking the CDR may be substituted or deleted, (ii) one or more amino acid residues may be added in one or more framework regions other than the framework regions flanking the CDR, or (iii) a combination of (i) and (ii), or (i) one or more amino acid residues in one or more of the flanking framework regions may be substituted or deleted, (ii) one or more amino acid residues may be added in one or more of the flanking framework regions, (iii) a combination of (i) and (ii); and (iv) one or more amino acid residues in one or more framework regions other than the framework regions flanking the CDR may be substituted or deleted, (v) one or more amino acid residues may be added in one or more framework regions other than the framework regions flanking the CDR, or (vi) a combination of (iv) and (v).

In a specific embodiment, the variable region contains one or more cysteine residues that form at least one intrachain disulfide bond, and at least one of said disulfide bonds is disrupted.

The invention also provides molecules comprising the variants described herein. The molecules can include one or more constant domains from an immunoglobulin; a second variable domain associated with the variant such as, for example, a variable domain of a heavy chain is associated with a variable domain of a light chain in an immunoglobulin; and a second variable domain associated with the variant, with one or more constant domains from immunoglobulins.

Also provided are immunoglobulins comprising a heavy chain and a light chain, wherein said heavy chain comprises a variant as described above and three constant domains from an immunoglobulin heavy chain, and the light chain comprises a second variable domain associated with the variant and a constant domain from an immunoglobulin light chain. Furthermore, the present invention provides immunoglobulins comprising a heavy chain and a light chain, wherein the light chain comprises a variant as described above and a constant domain from an immunoglobulin light chain, and the heavy chain comprises a second variable domain associated with said variant and three constant domains from an immunoglobulin heavy chain.

Isolated nucleic acids encoding these variants, molecules, and immunoglobulins are also provided, as are cells containing these nucleic acids. Recombinant non-human hosts containing these nucleic acids are also provided. Pharmaceutical compositions comprising a therapeutically or prophylactically effective amount of the variants, molecules or immunoglobulins and pharmaceutically acceptable carriers are also provided, as are vaccines comprising an effective amount of the variants, molecules, or immunoglobulin to induce an immune response. Vaccines comprising an effective amount of the variants, molecules, and immunoglobulin to induce an anti-idiotypic response are also provided. Preferably in the vaccines, the variant, molecule, or immunoglobulin has a disrupted intrachain disulfide bond in the modified variable region.

Methods are also provided for treating or preventing a disease in a subject in need of such treatment or prevention, which comprise administering to the subject a disease treating or preventing effective amount of a variant, molecule, or immunoglobulin wherein (i) the disease is caused directly or indirectly by an agent, (ii) a symptom of the disease is caused by an agent, or (iii) the disease produces a physical, chemical, or biological response, wherein the agents or response include said target sequence or molecule.

In a preferred embodiment, the invention provides a synthetic antibody that contains, in a CDR, the peptide sequence from angiogenin that confers binding to its receptor. This CDR is flanked by framework regions of a variable region. This synthetic antibody may be modified such that a cysteine residue that forms an intrachain disulfide bond in the variable region is disrupted, e.g., substituted or eliminated. A preferred synthetic antibody is a modified immunoglobulin wherein the framework region of the initial immunoglobulin contains cysteine residues that from an intrachain disulfide bond in said variable region, and one or more of said cysteine residues is disrupted.

In a further embodiment, the invention provides a nucleic acid encoding these constructs.

Another embodiment of the invention provides pharmaceutical compositions that comprise a therapeutically or prophylactically effective amount of the variant, molecule, or immunoglobulin, e.g. synthetic antibody, or an amount of a nucleic acid vector encoding any of the foregoing constructs. For example, such an amount is effective to inhibit binding of angiogenin to actin in vivo or in vitro. These compositions may further include a pharmaceutically acceptable carrier or excipient. The invention also provides a vaccine composition comprising an immune response or an anti-idiotypic response effective amount of the variant, molecule, or immunoglobulin, e.g., synthetic antibody, or nucleic acid vector encoding the construct. Preferably, these proteins include a disrupted intrachain disulfide bond. This vaccine can further include an adjuvant.

Preferably, the variant, molecule, or immunoglobulin, e.g. synthetic antibody, comprises one or more sequences ENKNGNPHRENLR (SEQ ID NO:4), preferably in CDR2 of a human heavy chain variable region or DNA which encodes the same.

Also encompassed are expression vectors, in which the nucleic acid encoding the construct is operably associated with an expression control sequence. The invention extends to host cells transfected or transformed with the expression vector. The constructs or DNA can be produced by isolating them from the host cells grown under conditions that permit production of the nucleic acid or expression of the construct. Alternatively, as disclosed above, the vectors themselves can be used as delivery vehicles of the construct in vivo or ex vivo.

The pharmaceutical and vaccine compositions of the invention can be administered to a subject to inhibit angiogenesis.

### DESCRIPTION OF THE DRAWINGS

FIGS. 1A and 1B. Consensus sequences of (A) the heavy chain variable region and (B) the light variable region.

FIG. 2. Diagram of PCR knitting strategy.

FIG. 3. AngioH2 (where the angiogenin sequence, ENKNGNPHRENLR (SEQ ID NO:4), has been inserted into CDR2 of the heavy chain variable region) binds to the angiogenin receptor actin. One of either three angiogenin heavy chain or three light chain constructs was added to individual wells of actin coated plates at 1 μg/ml. Consensus antibody was used at the same concentration as control. The binding was detected by using goat-anti-human IgG-HRP conjugate plus substrate. One construct. AngioH2, demonstrated potent binding to actin.

FIG. 4. The dose response of AngioH2 binding to actin was determined by ELISA. Various concentrations of AngioH2 were added to wells of actin-coated plates (solid triangles). Consensus antibody was used at the same concentrations as control (solid squares). The binding was detected by using goat-anti-human IgG-HRP conjugate plus substrate. AngioH2 bound to actin in a concentration-dependent manner.

FIG. 5. AngioH2 antagonizes the binding of angiogenin to actin. Recombinant human angiogenin (36 nM, EC₅₀) was mixed with various concentrations of AngioH2 (solid triangles) or consensus antibody (solid squares) and added to an actin coated plate. The binding was detected by using anti-angiogenin antibody-HRP conjugate plus substrate. AngioH2 antagonized angiogenin binding to actin in a concentration-dependent manner (Kᵢ˜4.2 nM).

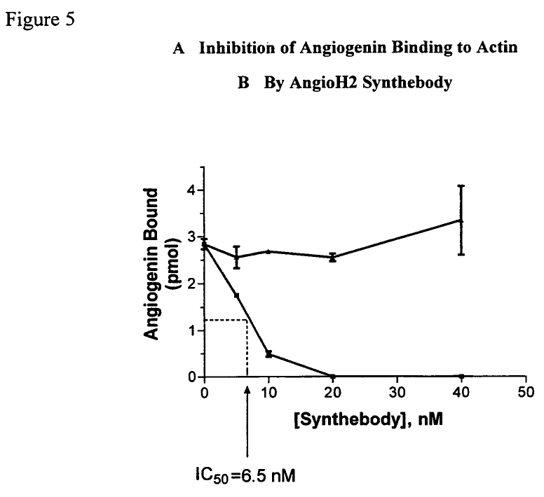

### DETAILED DESCRIPTION OF THE INVENTION

The present invention provides two approaches to the inhibition of angiogenesis using constructs that incorporate a receptor-binding portion of angiogenin in an immunoglobulin variable domain to create a variant of an immunoglobulin variable domain. In one aspect, a variant of an immunoglobulin variable domain, or a molecule which includes such a variant, such as an immunoglobulin or synthetic antibody, competitively inhibits angiogenin binding to its receptor, e.g. actin. In another aspect, a variant or molecule that elicits a potent anti-idiotype response can be used to generate an angiogenin inhibitory immune response.

Thus, in one aspect, the invention provides a variant of an immunoglobulin variable domain, or molecule comprising such a variant, including, but not limited to, an immunoglobulin molecule, that contains an amino acid sequence that can bind to a target sequence or molecule, such as, for example, a receptor-binding portion of angiogenin, in a CDR, which CDR is flanked by framework regions of a variable region. In a specific embodiment, this construct is modified such that an intrachain disulfide bond in the variable region is disrupted, e.g., one or more cysteine residues are substituted with a non-disulfide-forming amino acid, or eliminated. In this latter embodiment, the construct is particularly effective in generating an anti-idiotype response. The invention further provides pharmaceutical compositions that comprise an amount of the construct effective to inhibit binding of angiogenin and actin in vivo or a therapeutically or prophylactically effective amount of the construct and a pharmaceutically acceptable carrier or excipient. In a more specific embodiment, the invention also provides a vaccine composition comprising an immunogenic (immune response inducing) amount of the construct with a disrupted intrachain disulfide bond and an adjuvant.

Recombinant nucleic acids, particularly DNA molecules, provide for efficient expression of the foregoing constructs. In one aspect of this embodiment, the invention provides a nucleic acid encoding the construct. In a further aspect, the invention provides a nucleic acid encoding the construct with one or more disrupted intrachain disulfide bonds. Also encompassed are expression vectors in which the nucleic acid is operably associated with an expression control sequence. The invention extends to host cells transfected or transformed with the expression vector. The construct can be produced by isolating it from the host cells grown under conditions that permit expression of the construct.

The invention also furnishes a pharmaceutical composition comprising the expression vector that expresses the construct in an amount effective to express sufficient construct to inhibit binding of angiogenin and actin in vivo, and a pharmaceutically acceptable carrier or excipient. In a more specific embodiment, the invention concerns vaccine compositions comprising the expression vector for the disrupted intrachain disulfide bond construct in an amount effective to express an immunogenic amount of the construct, preferably in combination with an adjuvant or immunostimulatory agent.

The pharmaceutical and vaccine compositions of the invention can be administered to a subject to inhibit angiogenesis for the treatment of cancer, rheumatoid arthritis, diabetic retinopathy, psoriasis, hemangiomas, and arteriovenous malformations.

The major clinical indication for the angiogenin constructs is the adjuvant treatment of solid tumors. Additional potential indications include, but are not limited to, rheumatoid arthritis, diabetic retinopathy, endometriosis, and psoriasis. In addition, the constructs of the invention are clearly useful as diagnostic or analytical reagents for detecting actin in vitro and in vivo.

The present invention is based, in part, on the development of an actin-binding synthetic antibody variable domain by insertion of a receptor-binding portion of angiogenin (preferably corresponding to residues 58 to 70 of the full length polypeptide) into CDR2 of a consensus heavy chain variable region. This construct binds to actin with high affinity. In addition, it blocks the interaction between angiogenin and actin, i.e., it inhibits angiogenin binding to actin. Optimally, it mediates suppression of angiogenisis-dependent tumor growth in vivo.

In addition to direct blockade of angiogenin's binding, the construct can induce immune responses that further interfere with the angiogenesis process and suppress tumor growth. Thus, an anti-idiotype vaccine can also be developed using an angiogenin construct. Both Ab2 and Ab3 generated by the vaccine can block the interaction between angiogenin and actin (Ab2 will target angiogenin and Ab3 will target actin) and thereby become a sustained source of anti-angiogenesis activity. In specific embodiments, constructs are used to make anti-idiotype constructs by replacing the intrachain-disulfide bond forming cysteines with alanines in the variable region.

The term “construct” refers to the variant of a variable domain of an immunoglobulin superfamily protein, including molecules comprising such variants, described herein. The immunoglobulin superfamily is well known, and includes antibody/B-cell receptor proteins, T cell receptor proteins, and other proteins mentioned infra (see, Paul, Fundamental Immunology, 3^{rd} Ed.) The modification refers to insertion into or substitution of a portion of the immunoglobulin superfamily protein sequence with a heterologous amino acid sequence or heterologous binding sequence. The site of substitution in the immunoglobulin superfamily protein corresponds to a binding-accessible portion of the region of the immunoglobulin superfamily protein, e.g., a region that corresponds to an antibody variable region, and more particularly a portion corresponding to a CDR of an antibody variable region.

A “synthebody” (for synthetic antibody) is a specific example of a construct of the invention, which includes an antibody variable region. It may also include regions corresponding to an antibody constant region or regions, or be associated with one or more other immunoglobulin family polypeptides, such as an antibody Fv heterodimer, an antibody tetramer, a T cell receptor heterodimer, etc. Embodiments described below are illustrative of the variants and molecules of the present invention in that the variants are included in synthebodies and synthebodies are a type of molecule that includes the variants. The term “synthebody” thus refers to an illustrative example of a type of construct of the invention.

The term “heterologous” refers to a combination of elements not naturally occurring in a particular locus. For example, heterologous DNA refers to DNA not naturally located in the cell or in a particular chromosomal site of the cell. A heterologous expression regulatory element is such an element operatively associated with a different gene than the one it is operatively associated with in nature. In the context of the present invention, a construct coding sequence is heterologous to the vector DNA in which it is inserted for cloning or expression and it is heterologous to a host cell containing such a vector in which it is expressed, e.g., a CHO cell. Moreover, the constructs of the present invention contain a heterologous DNA, amino acid, or binding sequence.

The “heterologous amino acid (or binding) sequence” refers to the segment of a polypeptide, e.g., the portion of a polypeptide (protein or peptide) that binds to a target sequence or molecule, such as, for example, receptor. The sequence of the actin-binding site of angiogenin is a heterologous binding sequence.

A “target” is a molecule that is recognized by and specifically bound by the molecule from which the heterologous binding sequence is derived (in this case, angiogenin), and which therefore is recognized and specifically bound by a construct. In particular, a target can be a target receptor such as actin.

The term “CDR” refers to a part of the variable region of an immunoglobulin family protein that confers binding specificity, e.g. antibody specificity for antigen. In antibodies, CDRs are highly variable. For the immunoglobulin superfamily in general, the CDR or regions of the immunoglobulin domain corresponding to the CDR are accessible in the native folded conformational state. The site of introduction of the heterologous sequence is termed herein a “CDR”.

The term “framework region” refers to the part of the construct corresponding to an antibody framework region, as defined in the art. Sequences flanking the CDR are framework regions of a variable region domain that flank a particular CDR.

The term “flanked” or “flanking” refers to the amino acids that are connected to or are connected by spacing amino acids to the protein sequence of the CDR. “Spacing amino acids” (or a “spacer group”) are amino acids that are not found in the native framework sequence or the CDR or the substituted sequence, nor do they independently confer any binding activity on the modified variable region. They may be included to preserve or ensure a proper variable region conformation and orientation of the CDR or substituted heterologous amino acid sequence.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramvl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, cholera toxin, BCG (bacille Calmette-Guerin) and Corynebacterium parvum. Preferably, the adjuvant is pharmaceutically acceptable.

The phrase “pharmaceutically acceptable”, whether used in connection with the pharmaceutical compositions of the invention or vaccine compositions of the invention, refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The term “about” or “approximately” will be known to those skilled in the art in light of this disclosure. Preferably, the term means within 20%, more preferably within 10%, and more preferably still within 5% of a given value or range. Alternatively, especially in biological systems, the term “about” preferably means within about a log (i.e. an order of magnitude) preferably within a factor of two of a given value, depending on how quantitative the measurement.

### Molecular Biology—Definitions

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins, and may or may not include regulatory DNA sequences, such as promoter sequences, that determine for example the conditions under which the gene is expressed. The transcribed region of a gene can include 5′- and 3′-untranslated regions (UTRs) and introns in addition to the translated (coding) region.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of or “operably associated with” transcriptional and translational control sequences in a cell when RNA polymerase transcribes the coding sequence into mRNA, which is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as an mRNA or a protein. The expression product itself, e.g. the resulting mRNA or protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell from somewhere on or inside the cell. “Conditions that permit expression” in vitro are culture conditions of temperature (generally about 37° C.), humidity (humid atmosphere), carbon dioxide concentration to maintain pH (generally about 5% CO, to about 15% CO₂), pH (generally about 7.0 to 8.0, preferably 7.5), and culture fluid components that depend on host cell type. In vivo, the conditions that permit expression are primarily the health of the non-human transgenic animal, which depends on adequate nutrition, water, habitation, and environmental conditions (light-dark cycle, temperature, humidity, noise level). In either system, expression may depend on a repressor or inducer control system, as well known in the art.

The term “transfection” means the introduction of a “foreign” (i.e. extrinsic or extracellular) gene. DNA or RNA sequence into a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transfected” and is a “transfectant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA segment that can be inserted into a vector or into another piece of DNA at a defined restriction site. Preferably, a cassette is an “expression cassette” in which the DNA is a coding sequence or segment of DNA that codes for an expression product that can be inserted into a vector at defined restriction sites. The cassette restriction sites generally are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid” that generally is a self-contained molecule of double-stranded DNA, usually of bacterial origin, that can readily accept additional (foreign) DNA and which can be readily introduced into a suitable host cell. A plasmid vector often contains coding DNA and promoter DNA and has one or more restriction sites suitable for inserting foreign DNA. A large number of vectors, including plasmid and fungal vectors, have been described for replication and/or expression in a variety of eukaryotic and prokaryotic hosts. Non-limiting examples include pKK plasmids (Amersharn Pharmacia Biotech), pUC plasmids, pET plasmids (Novagen. Inc. Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used, or manipulated in any way, for the production of a substance by the cell, for example the expression by the cell of a gene, a DNA or RNA sequence, a protein or an enzyme. Host cells can further be used for screening or other assays, as described infra. The host cell may be found in vitro, i.e., in tissue culture, or in vivo, i.e., in a microbe, plant or animal.

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. In a specific embodiment, the construct is expressed in COS-1 or CHO cells. Other suitable cells include NSO cells. HeLa cells. 293T (human kidney cells), mouse primary myoblasts, and NIH 3T3 cells.

The terms “mutant” and “mutation” mean any detectable change in genetic material, e.g., DNA, or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence. The term “variant” may also be used to indicate a modified or altered gene, DNA sequence, enzyme, cell, etc. i.e., any kind of mutant.

As used herein, the term “oligonucleotide” refers to a nucleic acid, generally of at least 10, preferably at least 15, and more preferably at least 20 nucleotides, preferably no more than about 100 nucleotides, that is hybridizable to a genomic DNA molecule, a cDNA molecule, or an mRNA molecule having a sequence of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the construct, or to detect the presence of nucleic acids encoding the construct. In a further embodiment, an oligonucleotide of the invention can form a triple helix with a construct-encoding DNA molecule, e.g., for purification purposes. Generally, oligonucleotides are prepared synthetically, preferably on a nucleic acid synthesizer. Accordingly, oligonucleotides can be prepared with non-naturally occurring phosphoester analog bonds, such as thioester bonds, etc.

### Constructs

The constructs of the invention can be derived from different sources, including any type of immunoglobulin superfamily protein molecule, for example, but not limited to, antibodies (immunoglobulins), T cell receptors, cell-surface adhesion molecules such as the co-receptors CD4, CD8, CD19. CD28, etc., and the invariant domains of MHC molecules. In a preferred embodiment of the invention, the construct is derived from an antibody, which can be any class of antibody, e.g., an IgG, IgE, IgM, IgD, or IgA. Preferably, the antibody is an IgG. Such antibodies may be in membrane bound (B-cell receptor) or secreted form, preferably secreted. Additionally, the antibody may be of any subclass of the particular class of antibodies. In another specific embodiment, the synthebody is derived from a T cell receptor.

CDR-grafted variable region encoding sequences (genes) have been constructed by various methods such as site-directed mutagenesis as described in Jones et al Nature, 1986, 321:522; Riechmann et al., Nature, 1988, 332:323: in vitro assembly of entire CDR-grafted variable regions (Queen et al., Proc. Natl. Acad. Sci. USA, 1989, 86:10029); and the use of PCR to synthesize CDR-grafted genes (Daugherty et al. Nucleic Acids Res. 1991, 19:2471). CDR-grafted antibodies are generated in which the CDRs of the murine monoclonal antibody are grafted onto the framework regions of a human antibody. Following grafting, most antibodies benefit from additional amino acid changes in the framework region to maintain affinity, presumably because framework residues are necessary to maintain CDR conformation, and some framework residues have been demonstrated to be part of the antigen combining site. Such CDR-grafted antibodies have been successfully constructed against various antigens, for example, antibodies against IL-2 receptor as described in Queen et al. (Proc. Natl. Acad. Sci. USA, 1989, 86:10029), antibodies against cell surface receptors-CAMPATH as described in Riechmann et al. (Nature, 1988, 332:323); antibodies against hepatitis B in Co et al. (Proc. Natl. Acad. Sci. USA, 1991, 88:2869); as well as against viral antigens of the respiratory syncitial virus in Tempest et al. (BioTechnology, 1991, 9:267). Thus, in specific embodiments of the invention, the construct comprises an immunoglobulin variable domain in which at least one of the framework regions has one or more amino acid residues that differ from the residue at that position in the naturally occurring framework region. The techniques employed in creating CDR-grafted antibodies and for modifying framework regions can be adapted for use in constructs of the invention.

The heterologous amino acid sequence can be inserted into any of the CDRs of the variable domain. It is within the skill in the art to insert the binding site into different CDRs of the variable domain and then screen the resulting construct for the ability to bind to the binding partner of the heterologous amino acid sequence. Thus, one can determine which CDR optimally contains the binding site. In specific embodiments, a CDR of either the heavy or light chain variable region is modified to contain the amino acid sequence of the binding site. In another specific embodiment, the construct contains a variable domain in which the first, second, or third CDR of the heavy chain variable region or the first, second, or third CDR of the light chain variable region contains the amino acid sequence of the binding site. In another embodiment of the invention, more than one CDR contains the amino acid sequence of the binding site or more than one CDR each contains a different binding site for the same molecule or contains a different binding site for a different molecule. In particular embodiments, two, three, four, five or six CDRs (per heavy chain-light chain pair) are engineered to contain the receptor-binding portion of angiogenin.

In specific embodiments of the invention, the binding site amino acid sequence is either inserted into the CDR without replacing any of the amino acid sequence of the CDR itself or, alternatively, the binding site amino acid sequence replaces all or a portion of the amino acid sequence of the CDR.

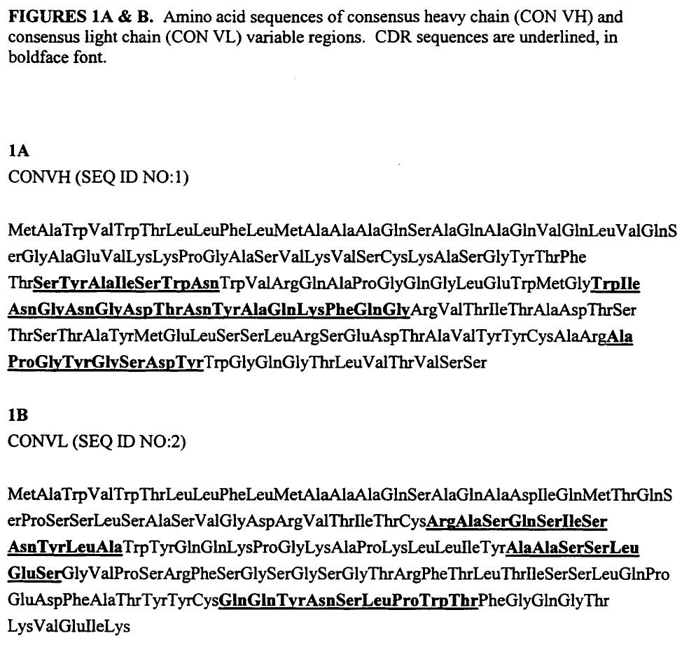

As shown in the Examples, infra, in a specific embodiment the heterologous binding sequence is very effective, compared to control, when inserted in CDR2 of the heavy chain variable region (SEQ ID NO:28). These results depend on the nature of the variable region framework structure, which was depicted in FIG. 1A (SEQ ID NO:51). A control construct comprises the original consensus variable region sequence.

Relative efficacy of an actin-binding construct can be evaluated by direct binding assays, such as ELISA, Western blotting, and the like; inhibition assays, e.g., with labeled angiogenin; and functional assays, including binding to endothelial cells, inhibition of angiogenesis in vivo or in vitro, or suppression of tumor growth in vivo.

After preparing constructs containing modified CDRs, the constructs can be further altered and screened to select an antibody having higher affinity or specificity. Constructs having higher affinity or specificity for the target antigen may be generated and selected by any method known in the art. For example, but not by way of limitation, the nucleic acid encoding the construct can be mutagenized, either randomly, i.e., by chemical or site-directed mutagenesis, or by making particular mutations at specific positions in the nucleic acid encoding the construct, and then screening the proteins expressed from the mutated nucleic acid molecules for binding affinity for the target. Screening can be accomplished by testing the expressed proteins individually or by screening a library of the mutated sequences, e.g., by phage display techniques (see, e.g., U.S. Pat. Nos. 5,223,409; 5,403,484; and 5,571,698; PCT Publication WO 92/01047) or any other phage display technique known in the art.

Accordingly, in a specific embodiment, the construct may have a higher specificity or affinity for its target antigen than a naturally occurring antibody that specifically binds the same target. In another embodiment, the construct exhibits a binding constant for target ranging from about 1×10⁶ to about 1×10¹⁴M⁻¹.

The construct of the invention may also be further modified in any way known in the art as long as the further modification does not completely prevent binding of the construct to the particular target. In particular, the constructs of the invention may have one or more amino acid substitutions, deletions, or insertions besides the insertion into or replacement of CDR sequences with the amino acid sequence of a binding sequence. Such amino acid substitutions, deletions, or insertions can be any substitution, deletion, or insertion that does not prevent the specific binding of the construct to the target binding partner. For example, such amino acid substitutions include substitutions of functionally equivalent amino acid residues. One or more amino acid residues can be substituted by another amino acid of a similar polarity that acts as a functional equivalent resulting in a silent alteration. Substitutes for an amino acid may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Additionally, one or more amino acid residues can be substituted by a nonclassical amino acid or chemical amino acid analogs, introduced as a substitution or addition into the sequence. Non-classical amino acids include but are not limited to the D (dextrorotary)-isomers of the common amino acids, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, beta-alanine, fluoro-amino acids, designer amino acids such as beta-methyl amino acids, C-alpha-methyl amino acids, N-alpha-methyl amino acids, and amino acid analogs in general.

### Anti-Idiotype Constructs

In a particular embodiment of the invention, the construct is further modified to enhance its ability to elicit an anti-idiotype response, for example, as described in PCT Publication No. WO 99/25379. Such modifications are made to reduce the conformational constraints on a variable domain, e.g., by removing or reducing intrachain disulfide bonds. Specifically, the construct is further modified such that one or more variable region cysteine residues that form disulfide bonds are replaced with an amino acid residue that does not have a sulfhydryl group.

Identifying the cysteine residues that form a disulfide bond in a variable region of a particular antibody can be accomplished by any method known in the art. For example, but not by way of limitation, it is well known in the art that the cysteine residues that form intrachain disulfide bonds are highly conserved among antibody classes and across species. Thus, the cysteine residues that participate in disulfide bond formation can be identified by sequence comparison with other antibody molecules in which it is known which residues form a disulfide bond (for example the consensus sequences provided in FIGS. 1A and 1B, or those described in Kabat et al., 1991, Sequences of Proteins of Immunological Interest, 5^{th} Ed., U.S. Department of Health and Human Services, Bethesda, Md.).

Notably, for most antibody molecules, the cysteine residues that form the intrachain disulfide bonds are residues at positions 23 and 88 of the light chain variable domain and residues at positions 22 and 92 of the heavy chain variable domain. The position numbers refer to the residue corresponding to that residue in the consensus sequences as defined in Kabat et al., supra, i.e. the “Kabat equivalent” or as indicated in the heavy and light chain variable region sequences depicted in FIGS. 1A and 1B, respectively (as determined by aligning the particular antibody sequence with the consensus sequence of the heavy or light chain variable region sequence depicted in FIGS. 1A and 1B).

Accordingly, in one embodiment of the invention, the construct is further modified such that the residues at positions 23 and/or 88 of the light chain as identified by Kabat et al., supra, or their equivalent, are substituted with an amino acid residue that does not contain a sulfhydryl group; and/or the residues at positions 22 and/or 92 are of the heavy chain as identified by Kabat et al. supra, or their equivalent, are substituted with an amino acid residue that does not contain a sulfhydryl group. As used in reference to intrachain disulfides, equivalents of the cysteines at the Kabat positions provided above are intrachain-forming cysteine residues at homologous positions in the immunoglobulin domain of an immunoglobulin superfamily protein molecule.

The amino acid residue that substitutes for the disulfide bond forming cysteine residue is any amino acid residue that does not contain a sulfhydryl group, e.g., alanine, arginine, asparagine, aspartate (or aspartic acid), glutamine, glutamate (or glutamic acid), glycine, histidine, isoleucine, leucine, lysine, phenylalanine, proline, serine, threonine, tryptophan, tyrosine or valine. In a preferred embodiment, the cysteine residue is replaced with a glycine, serine, threonine, tyrosine, asparagine, or glutamine residue, most preferably with an alanine residue.

Additionally, the disulfide bond forming cysteine residue may be replaced by a nonclassical amino acid or chemical amino acid analog, such as those listed supra, that does not contain a sulfhydryl group; or it may be chemically modified by reaction with the sulfhydryl to preclude disulfide bond formation.

In specific embodiments, the substitution of the disulfide bond forming residue is in the heavy chain variable region or the light chain variable region, or both the heavy chain and light chain variable regions. In other specific embodiments, one of the residues that forms a particular disulfide bond is replaced (or deleted) or, alternatively, both residues that form a particular disulfide bond may be replaced (or deleted).

### Immunoglobulin Fragment Constructs

As noted above, fragments of an immunoglobulin family protein can be modified to create a construct. For example, such fragments include but are not limited to: F(ab′)₂ fragments that contain the variable regions of both the heavy and the light chains, the light constant region and the CH1 domain of the heavy chain, which fragments can be generated by pepsin digestion of an antibody; Fab fragments, generated by reducing the disulfide bonds of an F(ab′)₂ fragment (King et al., Biochem. J. 1992, 281:317), and Fv fragments, i.e., fragments that contain the variable region domains of both the heavy and light chains (Reichmann and Winter. J. Mol. Biol. 1988, 203:825; King et al., Biochem, J. 1993, 290:723).

The present invention also includes, but is not limited to, single chain antibodies (SCA) (U.S. Pat. No. 4,946,778; Bird. Science, 1988, 242:423-426; Huston et al., Proc. Natl. Acad. Sci. USA, 1988, 85:5879-5883; and Ward et al. Nature, 1989, 334:544-546). Single chain antibodies are formed by linking the heavy and light chain fragments of the Fv region via an amino acid bridge, resulting in a single chain polypeptide. Additionally, the invention also provides heavy chain and light chain dimers and diabodies.

### Preferred Immunoglobulin Family Proteins for Constructs

Immunoglobulin molecules modified to generate constructs preferably are monoclonal antibodies. The antibody that is modified may be a naturally occurring or previously existing antibody, or may be synthesized from known antibody consensus sequences, such as the consensus sequences for the light and heavy chain variable regions in FIGS. 1A and 1B, or any other antibody consensus or germline (i.e., unrecombined genomic sequences) sequences (e.g., those antibody consensus and germline sequences described in Kabat et al., 1991, Sequences of Proteins of Immunological Interest, 5^{th} edition, NIH Publication No. 91-3242, pp. 2147-2172).

The invention further provides constructs that are also chimeric or humanized. For example, a chimeric antibody is a molecule in which different portions of the antibody molecule are derived from different animal species, such as those having a variable region derived from a murine mAb and a constant region derived from a human immunoglobulin constant region, e.g., humanized antibodies. Techniques have been developed for the production of chimeric antibodies (Morrison et al., Proc. Natl. Acad. Sci. USA. 1984. 81:6851-6855; Neuberger et al., Nature. 1984, 312:604-608; Takeda et al., Nature, 1985. 314:452-454; International Patent Application No. PCT/GB85/00392) by splicing the genes from a mouse antibody molecule of appropriate antigen specificity together with genes from a human antibody molecule of appropriate biological activity. In a specific embodiment, the construct is a chimeric antibody containing the variable domain of a non-human antibody and the constant domain of a human antibody.

In another embodiment, the construct is derived from a humanized antibody, in which the CDRs of the antibody (except for the one or more CDRs containing the heterologous binding sequence) are derived from an antibody of a non-human animal and the framework regions and constant region are from a human antibody (see. U.S. Pat. No. 5,225,539).

As noted above, the construct can be derived from a human monoclonal antibody. The creation of completely human monoclonal antibodies is possible through the use of transgenic mice. Transgenic mice in which the mouse immunoglobulin gene loci have been replaced with human immunoglobulin loci provide in vivo affinity-maturation machinery for the production of human immunoglobulins.

### Immunoglobulin Fusion Protein and Derivative Constructs

In certain embodiments, the construct is created by fusing (joining) an immunoglobulin family protein modified to include the heterologous binding sequence to an amino acid sequence of another protein (or portion thereof, preferably an at least 10, 20, or 50 amino acid portion thereof) that is not the modified immunoglobulin, thereby creating a fusion (or chimeric) construct. Preferably, the fusion is via covalent bond (for example, but not by way of limitation, a peptide bond) at either the N-terminus or the C-terminus. In preferred embodiments, the invention provides constructs in which the variant or molecule comprising the variant is covalently linked to a portion of an immunostimulatory factor, including, but not limited to, interleukin-2, interleukin-4, interleukin-5, interleukin-6, interleukin-7, interleukin-10, interleukin-12, interleukin-15, G-colony stimulating factor, tumor necrosis factor, porin, interferon-gamma, and NK cell antigen or MHC derived peptide.

The construct may be further modified, e.g., by the covalent attachment of any type of molecule, as long as such covalent attachment does not prevent or inhibit specific binding of the construct to its target antigen. For example, but not by way of limitation, the construct may be further modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc.

In specific embodiments of the invention, the construct is covalently linked to a therapeutic molecule, for example, to target the therapeutic molecule to a particular cell type or tissue, e.g., a cancer or tumor cell. The therapeutic molecule can be any type of therapeutic molecule known in the art, for example, but not limited to, a chemotherapeutic agent, a toxin, such as ricin, an antisense oligonucleotide, a radionuclide, an antibiotic, anti-viral, or anti-parasitic, etc.

### Structure of the Heterologous Binding (Amino Acid) Sequence

The mature protein for human angiogenin is a single chain, 123 amino acid polypeptide (Strvdom et al. Biochemistry, 1985, 24:5486). The sequence of angiogenin (from N-terminal to C-terrminal) is as follows:

QDNSRYTHFL TQHYDAKPQG RDDRYCESIM RRRGLTSPCK DINTFIHGNK RSIKAICENK NGNPHRENLR ISKSSFQVTT CKLHGGSPWP PCQYRATAGF RNVVVACENG LPVHLDQSIF RRP (SEQ ID NO:3)

Three disulfide bonds are known to exist, occurring between residues 26 and 81, 39 and 92, and 57 and 102 (Strydom et al., supra; Bond et al., Biochim. Biophy. Acta, 1993, 1162:177). In contrast to other ribonucleases, angiogenin lacks cysteines at analogous positions 64 and 69.

The actin binding site of angiogenin has been identified as amino acids 58-70 (ENKNGNPHRENLR; SEQ ID NO:4) (Hallahan, et al, Proc. Natl. Acad. Sci. USA 1991, 88:2222-2236) by mutagenesis and peptide substitution studies. It has been shown that replacement of RNase sequence 59-73 with the angiogenin peptide 58-70 enables the non-angiogenic protein to become angiogenic. Thus, in a preferred embodiment, the heterologous binding (amino acid) sequence of the construct of the invention is ENKNGNPHRENLR (SEQ ID NO:4), i.e. this is proposed to be the active binding portion of the construct.

### Methods of Producing the Constructs

The constructs of this invention can be produced by any method known in the art for the synthesis of immunoglobulins, in particular, by chemical synthesis or by recombinant expression, and are preferably produced by recombinant expression techniques.

Recombinant expression of the constructs of the invention requires construction of a nucleic acid encoding the construct. Such an isolated nucleic acid that contains a nucleotide sequence encoding the construct can be produced using any method known in the art.

In accordance with the present invention there may be employed conventional molecular biology, microbiology, and recombinant DNA techniques within the skill of the art. Such techniques are explained fully in the literature. See, e.g., Sambrook. Fritsch & Maniatis. Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization, B. D. Hames & S. J. Higgins eds. (1985); Transcription And Translation, B. D. Hames & S. J. Higgins, eds. (1984); Animal Cell Culture, R. I. Freshney, ed. (1986); Immobilized Cells And Enzymes, IRL Press, (1986); B. Perbal, A Practical Guide To Molecular Cloning (1984); F. M. Ausubel et al. (eds.). Current Protocols in Molecular Biology, John Wiley & Sons, Inc. (1994).

### Construct Nucleic Acid

Accordingly, the invention provides nucleic acids that contain a nucleotide sequence encoding a construct of the invention, or a functionally active fragment thereof.

A nucleic acid that encodes a construct may be assembled from chemically synthesized oligonucleotides (e.g., as described in Kutmeier et al., BioTechniques. 1994. 17:242), which, briefly, involves the synthesis of a set of overlapping oligonucleotides containing portions of the sequence encoding the construct, annealing and ligation of those oligonucleotides, and then amplification of the ligated oligonucleotides by PCR.

Accordingly, the invention provides a method of producing a nucleic acid encoding a construct, the method comprising: (a) synthesizing a set of oligonucleotides, the set comprising oligonucleotides containing a portion of the nucleotide sequence that encodes the construct and oligonucleotides containing a portion of the nucleotide sequence that is complementary to the nucleotide sequence that encodes the constructs, and each of the oligonucleotides having overlapping terminal sequences with another oligonucleotide of the set, except for those oligonucleotides containing the nucleotide sequences encoding the N-terminal and C-terminal portions of the construct; (b) allowing the oligonucleotides to hybridize or anneal to each other; and (c) ligating the hybridized oligonuclcotides, such that a nucleic acid containing the nucleotide sequence encoding the construct is produced.

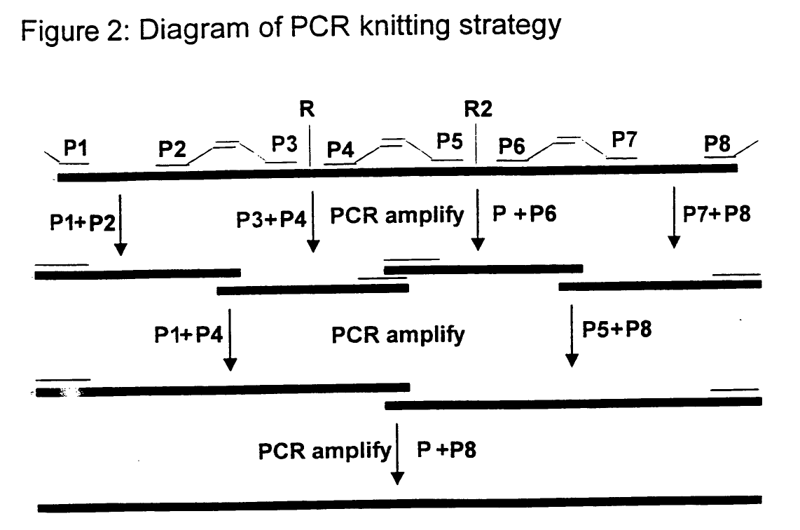

Another method for producing a nucleic acid encoding a construct is to modify nucleic acid sequences that encode an antibody molecule (or other immunoglobulin superfamily protein), or at least the variable region thereof, using the “PCR knitting” approach (FIG. 2). In “PCR knitting”, nucleic acid sequences, such as the consensus variable region sequences shown in Example 1, are used as templates for a series of PCR reactions that result in the selective insertion of a nucleotide sequence that encodes the desired peptide sequence (in this example, the actin binding sequence of angiogenin) into one or more CDRs of the variable region. Oligonucleotide primers are designed for these PCR reactions that contain regions complementary to the framework sequences flanking the designated CDR at the 3′ends and sequences that encode the peptide sequence to be inserted at the 5′ends. In addition, these oligonucleotides contain approximately ten bases of complementary sequences at their 5′ends. These oligonucleotide primers can be used with additional flanking primers to insert the desired nucleotide sequence into the selected CDR as shown in FIG. 2 resulting in the production of a nucleic acid coding for the variant of the variable domain.

Alternatively, a nucleic acid containing a nucleotide sequence encoding a construct can be constructed from a nucleic acid containing a nucleotide sequence encoding, e.g., an antibody molecule, or at least a variable region of an antibody molecule. Nucleic acids containing nucleotide sequences encoding antibody molecules can be obtained either from existing clones of antibody molecules or variable domains or by isolating a nucleic acid encoding an antibody molecule or variable domain from a suitable source, preferably a cDNA library, e.g., an antibody DNA library or a cDNA library prepared from cells or tissue expressing a repertoire of antibody molecules or a synthetic antibody library (see, e.g., Clackson et al., Nature. 1991, 352:624; Hane et al., Proc. Natl. Acad. Sci. USA. 1997, 94:4937), for example, by hybridization using a probe specific for the particular antibody molecule or by PCR amplification using synthetic primers hybridizable to the 3′ and 5′ ends of the sequence.

If a convenient restriction enzyme site is available in the nucleotide sequence of the CDR, then the sequence can be cleaved with the restriction enzyme and a nucleic acid fragment containing the nucleotide sequence encoding the binding site can be ligated into the restriction site. The nucleic acid fragment containing the binding site can be obtained either from a nucleic acid encoding all or a portion of the protein containing the binding site or can be generated from synthetic oligonucleotides containing the sequence encoding the binding site and its reverse complement.

The nucleic acid encoding the construct optionally contains a nucleotide sequence encoding a leader sequence that directs the secretion of the construct.

### Construct Expression

Once a nucleic acid encoding a construct is obtained, it may expressed, or it may be introduced into a vector containing the nucleotide sequence encoding the constant region of the antibody (see, e.g., PCT Publications WO 86/05807 and WO 89/01036; and U.S. Pat. No. 5,122,464). Vectors containing the complete light or heavy chain for co-expression are available to allow the expression of a complete antibody molecule and are known in the art, for example, pMRRO10.1 and pGammal (see also, Bebbington, In Methods: a companion to Methods in Enzymology, 1991, 2:136-145). Alternatively, variant variable domain can be introduced into a construct for expression of a chimeric molecule.

The expression vector can then be transferred to a host cell in vitro or in vivo by conventional techniques and the transfected cells can be cultured by conventional techniques to produce the antibody of the invention. Specifically, once a variant of an immunoglobulin variable domain has been generated, the construct can be expressed, for example, by the method exemplified in the Examples (see also Bebbington, supra). For example, transiently transfecting the expression vector encoding a construct into COS cells, culturing the cells for an appropriate period of time to permit construct expression, and then taking the supernatant from the COS cells, which supernatant contains the secreted, results in production of the construct.

The host cells used to express the recombinant construct may be either bacterial cells such as Escherichia coli, particularly for the expression of recombinant antibody fragments or, preferably, eukaryotic cells, particularly for the expression of recombinant immunoglobulin molecules, in particular, mammalian cells such as Chinese hamster ovary cells (CHO) or COS cells, used in conjunction with a vector in which expression of the construct is under control of the major intermediate early gene promoter element from human cytomegalovirus, is an effective expression system for immunoglobulins (Foecking et al. Gene, 1986, 45:101; Cockett et al., BioTechnology, 1990, 8:662).

A variety of host-expression vector systems may be utilized to express the construct of the invention. Such host-expression systems represent vehicles by which the products of interest may be produced and subsequently purified, but also may be used to transform or transfect cells with the appropriate nucleotide coding sequences to produce the product of the invention in situ. These systems include, but are not limited to, microorganisms such as bacteria (e.g., E. coli, B. subtilis) transformed with recombinant bacteriophage DNA, plasmid DNA or cosmid DNA expression vectors containing construct coding sequences; yeast (e.g., Saccharomyces, Pichia) transformed with recombinant yeast expression vectors containing construct coding sequences; insect cell systems infected with recombinant virus expression vectors (e.g., baculovirus) containing the antibody coding sequences; plant cell systems infected with recombinant virus expression vectors (e.g. cauliflower mosaic virus, CaMV; tobacco mosaic virus, TMV) or transformed with recombinant plasmid expression vectors (e.g., Ti plasmid) containing antibody coding sequences; mammalian cell systems (e.g., COS, CHO, BHK, 293, and 3T3 cells) harboring recombinant expression constructs containing promoters derived from the genome of mammalian cells (e.g., the metallothionein promoter) or from mammalian viruses (e.g., the adenovirus late promoter; the vaccinia virus 7.5K promoter); and transgenic animal systems, particularly for expression in milk (e.g., U.S. Pat. Nos. 5,831,141 and 5,849,992, which describe transgenic production of antibodies in milk; U.S. Pat. No. 4,873,316).

Expression of the protein or polypeptide may be controlled by any promoter/enhancer element known in the art, but these regulatory elements must be functional in the host selected for expression. Promoters that may be used to control gene expression include, but are not limited to, cytomegalovirus (CMV) promoter (U.S. Pat. Nos. 5,385,839 and 5,168,062), the SV40 early promoter region (Benoist and Chambon, Nature, 1981, 290:304-310), the promoter contained in the 3′ long terminal repeat of Rous sarcoma virus (Yamamoto, et al., Cell, 1980, 22:787-797), the herpes thymidine kinase promoter (Wagner et al., Proc. Natl. Acad. Sci. U.S.A., 1981, 78:1441-1445), the regulatory sequences of the metallothionein gene (Brinster et al, Nature, 1982, 296:39-42); prokaryotic expression vectors such as the B-lactamase promoter (Villa-Komaroff, et al., Proc. Natl. Acad. Sci. U.S.A., 1978, 75:3727-3731), or the tac promoter (DeBoer, et al., Proc. Natl. Acad. Sci. U.S.A., 1983, 80:21-25); see also “Useful proteins from recombinant bacteria” in Scientific American, 1980, 242:74-94; promoter elements from yeast or other fingi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature, 1985, 315:338-340; Kollias et al., 1986, Cell 46:89-94), hematopoietic stem cell differentiation factor promoters, erythropoietin receptor promoter (Maouche et al., Blood, 1991, 15:2557), etc.

In bacterial systems, a number of expression vectors may be advantageously selected depending upon the use intended for the construct being expressed. For example, when a large quantity of such a protein is to be produced, for the generation of pharmaceutical compositions of a construct, vectors which direct the expression of high levels of fusion protein products that are readily purified may be desirable. Such vectors include, but are not limited to, the E. coli expression vector pUR278 (Ruther et al., EMBO J., 1983, 2:1791), in which the coding sequence may be ligated individually into the vector in frame with the lac Z coding region so that a fusion protein is produced; pIN vectors (Inouye & Inouye, Nucleic Acids Res., 1985, 13:3101-3109; Van Hleeke & Schuster, J. Biol. Chem., 1989, 264:5503-5509); and the like, pGEX vectors may also be used to express foreign polypeptides as fusion proteins with glutathione S-transferase (GST). In general, such fusion proteins are soluble and can easily be purified from lysed cells by adsorption and binding to a matrix of glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of viral-based and non-viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the construct in infected hosts (see, e.g., Logan and Shenk, Proc. Natl. Acad. Sci. U.S.A., 1984, 81:3655-3659). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al. Methods in Enzymol., 1987, 153:516-544).

Additionally, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138.

For long-term, high-yield production of recombinant proteins, stable expression is preferred. For example, cell lines that stably express the antibody may be engineered. Rather than using expression vectors that contain viral origins of replication, host cells can be transformed with DNA controlled by appropriate expression control elements (e.g., promoter, enhancer sequences, transcription terminators, polyadenylation sites, etc.) and a selectable marker. Following the introduction of the foreign DNA, engineered cells may be allowed to grow for 1-2 days in an enriched media., and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the antibody. Such engineered cell lines may be particularly useful in screening and evaluation of compounds that interact directly or indirectly with the antibody.

A number of selection systems may be used, including but not limited to the herpes simplex virus thyrmidine kinase (Wigler et al., Cell, 1977, 11:223), hypoxanthine-guanine phosphoribosyltransferase (Szvbalska and Szvbalski. Proc. Natl. Acad. Sci. USA. 1962,48:2026), and adenine phosphoribosyltransferase (Lowy et al., Cell, 1980, 22:817) genes can be employed in tk-, hgprt-, or aprt-cells, respectively. Also, antimetabolite resistance can be used as the basis of selection for the following genes: dhfr, which confers resistance to methotrexate (Wigler et al., Proc. Natl. Acad. Sci. USA, 1980, 77:3567; O'Hare et al., Proc. Natl. Acad. Sci. USA, 1981, 78:1527); gpt, which confers resistance to mycophenolic acid (Mulligan and Berg, Proc. Natl. Acad. Sci. USA, 1981, 78:2072); neo, which confers resistance to the aminoglycoside G418 (Colberre-Garapin et al. J. Mol. Biol. 1981, 150:1); and hygro, which confers resistance to hygromycin (Santerre et al. Gene, 1984. 30:147).

The expression levels of the construct can be increased by vector amplification (for a review, see Bebbington and Hentschel. The Use of Vectors Based on Gene Amplification for the Expression of Cloned Genes in Mammalian Cells in DNA Cloning, Vol. 3., Academic Press. New York, 1987). When a marker in the vector system expressing a construct is amplifiable, increases in the level of inhibitor present in the culture medium of the host cell will increase the number of copies of the marker gene. Since the amplified region is associated with the construct encoding gene, production of the construct will also increase (Crouse et al., Mol. Cell. Biol., 1983, 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, Nature, 1986, 322:562; Kohler. Proc. Natl. Acad. Sci. USA, 1980, 77:2197). The coding sequences for the heavy and light chains may comprise cDNA or genomic DNA.

### Viral and Non-Viral Vectors

Preferred vectors, particularly for expression in vitro and in vivo, are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication No. WO 95/28494.

Viral vectors commonly used for in vivo or ex vivo targeting and therapy procedures are DNA-based vectors and retroviral vectors. Methods for constructing and using viral vectors are known in the art (see, e.g. Miller and Rosman, BioTechniques, 1992, 7:980-990). Preferably, the viral vectors are replication-defective, that is, they are unable to replicate autonomously in the target cell. Preferably, the replication defective virus is a minimal virus, i.e. it retains only the sequences of its genome which are necessary for encapsidating the genome to produce viral particles.

DNA viral vectors include an attenuated or defective DNA virus, such as but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV 1) vector (Kaplitt et al., Molec. Cell. Neurosci., 1991, 2:320-330), defective herpes virus vector lacking a glyco-protein L gene, or other defective herpes virus vectors (PCT Publication Nos. WO 94/21807 and WO 92/05263); an attenuated adenovirus vector, such as the vector described by Stratford-Perricaudet et al. (J. Clin. Invest., 1992, 90:626-630; see also La Salle et al., Science, 1993, 259:988-990); and a defective adeno-associated virus vector (Samulski et al., J. Virol., 1987, 61:3096-3101; Samulski et al., J. Virol., 1989, 63:3822-3828; Lebkowski et al., Mol. Cell. Biol., 1988, 8:3988-3996).

Various companies produce viral vectors commercially, including, but not limited to. Avigen. Inc. (Alameda, Calif.; AAV vectors), Cell Genesys (Foster City, Calif.; retroviral, adenoviral, AAV, and lentiviral vectors), Clontech (retroviral and baculoviral vectors), Genovo, Inc. (Sharon Hill, Pa.; adenoviral and AAV vectors), Genvec (France; adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France: adenoviral, vaccinia, retroviral, and lentiviral vectors).

Adenovirus vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5 or adenoviruses of animal origin (see PCT Publication No. WO 94/26914). Those adenoviruses of animal origin which can be used within the scope of the present invention include adenoviruses of canine, bovine, murine (example: Mavl, Beard et al., Virology, 1990. 75-81), ovine, porcine, avian, and simian (example: SAV) origin. Preferably, the adenovirus of animal origin is a canine adenovirus, more preferably a CAV2 adenovirus (e.g., Manhattan or A26/61 strain. ATCC VR-800, for example). Various replication defective adenovirus and minimum adenovirus vectors have been described (PCT Publication Nos. WO 94/26914, WO 95/02697, WO 94/28938, WO 94/28152, WO 94/12649, WO 95/02697, WO 96/22378). The replication defective recombinant adenoviruses according to the invention can be prepared by any technique known to the person skilled in the art (Levrero et al., Gene, 1991, 101:195; European Publication No. EP 185 573; Graham, EMBO J., 1984, 3:2917; Graham et al., J. Gen. Virol., 1977, 36:59). Recombinant adenoviruses are recovered and purified using standard molecular biological techniques, which are well known to one of ordinary skill in the art.

Adeno-associated viruses. The adeno-associated viruses (AAV) are DNA viruses of relatively small size that can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see, PCT Publication Nos. WO 91/18088 and WO 93/09239; U.S. Pat. Nos. 4,797,368 and 5,139,941; European Publication No. EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

Retrovirus vectors. In another embodiment the gene can be introduced in a retroviral vector, e.g., as described in U.S. Pat. No. 5,399,346: Mann et al., Cell, 1983. 33:153; U.S. Pat. Nos. 4,650,764 and 4,980,289; Markowitz et al. J. Virol., 1988, 62:1120; U.S. Pat. No. 5,124,263; European Publication Nos. EP 453 242 and EP 178 220; Bernstein et al., Genet. Eng., 1985, 7:235; McCormick, BioTechnology, 1985, 3:689; PCT Publication No. WO 95/07358; and Kuo et al., Blood, 1993, 82:845. The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus”); MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (PCT Publication No. WO 90/02806) and the GP+envAm-12 cell line (PCT Publication No. WO 89/07150). In addition, the recombinant retroviral vectors can contain modifications within the LTRs for suppressing transcriptional activity as well as extensive encapsidation sequences which may include a part of the gag gene (Bender et al., J. Virol., 1987, 61:1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retrovirus vectors can also be introduced by DNA viruses, which permits one cycle of retroviral replication and amplifies tranfection efficiency (see PCT Publication Nos. WO 95/22617, WO 95/26411, WO 96/39036 and WO 97/19182).

Letitivirus vectors. In another embodiment, lentiviral vectors can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini, Curr. Opin. Biotechnol., 1998, 9:457-63; see also Zufferey, et al., J. Virol., 1998, 72:9873-80). Lentiviral packaging cell lines are available and known generally in the art. They facilitate the production of high-titer lentivirus vectors for gene therapy. An example is a tetracycline-inducible VSV-G pseudotyped lentivirus packaging cell line that can generate virus particles at titers greater than 10⁶ IU/ml for at least 3 to 4 days (Kafri, et al., J. Virol., 1999, 73: 576-584). The vector produced by the inducible cell line can be concentrated as needed for efficiently transducing non-dividing cells in vitro and in vivo.

Non-viral vectors. In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A., 1987, 84:7413-7417; Felgner and Ringold, Science, 1989, 337:387-388; see Mackey, et al., Proc. Natl. Acad. Sci. U.S.A., 1988, 85:8027-8031; Ulmer et al., Science, 1993, 259:1745-1748). Useful lipid compounds and compositions for transfer of nucleic acids are described in PCT Patent Publication Nos. WO 95/18863 and WO 96/17823, and in U.S. Pat. No. 5,459,127. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Publication No. WO 95/21931), peptides derived from DNA binding proteins (e.g., PCT Patent Publication No. WO 96/25508), or a cationic polymer (e.g., PCT Patent Publication No. WO 95/21931).

It is also possible to introduce the vector in vivo as a naked DNA plasmid. Naked DNA vectors for gene therapy can be introduced into the desired host cells by methods known in the art, e.g., electroporation, microinjection, cell fusion. DEAE dextran, calcium phosphate precipitation, use of a gene gun, or use of a DNA vector transporter (see, e.g., Wu et al., J. Biol. Chem. 1992, 267:963-967. Wu and Wu, J. Biol. Chem., 1988, 263:14621-14624; Canadian Patent Application No. 2,012,311; Williams et al., Proc. Natl. Acad. Sci. USA, 1991, 88:2726-2730). Receptor-mediated DNA delivery approaches can also be used (Curiel et al., Hum. Gene Ther., 1992, 3:147-154; Wu and Wu. J. Biol. Chem., 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci., 1988, 321:893; PCT Publication Nos. WO 99/01157; WO 99/01158; WO 99/01175).

### Therapeutic Use of Constructs

The invention also provides methods for treating or preventing diseases and disorders associated with the activity of a particular molecule (angiogenin) by administration of a therapeutic of the invention. Such therapeutics include the constructs of the invention and nucleic acids encoding the constructs of the invention.

Generally, administration of products of a species origin or species reactivity that is the same species as that of the recipient is preferred. Thus, in administration to humans, the therapeutic methods of the invention use a construct that is preferably derived from a human immunoglobulin, but may be an immunoglobulin from a heterologous species such as, for example, a mouse, which may or may not be humanized. In other embodiments, the methods of the invention use a modified immunoglobulin that is derived from a chimeric or humanized immunoglobulin.

Specifically, pharmaceutical compositions containing the constructs of the invention that specifically bind a particular molecule can be used in the treatment or prevention of diseases or disorders associated with the expression of the particular molecule, e.g., binding partner. In particular, in embodiments discussed in more detail in the subsections that follow, constructsd that specifically bind actin can be used to treat various conditions, such as solid tumors.

The subjects to which the present invention is applicable may be any mammalian or vertebrate species, which include, but are not limited to, cows, horses, sheep, pigs, fowl (e.g., chickens), goats, cats, dogs, hamsters, mice, rats, monkeys, rabbits, chimpanzees, and humans. In a preferred embodiment, the subject is a human.

### Treatment and Prevention of Cancers

The invention provides methods of treating or preventing cancers. The method includes administering to a subject in need of such treatment or prevention a therapeutic of the invention, i.e. a construct, that specifically binds to actin, which construct comprises a variable domain with a CDR containing the amino acid sequence of a binding site for actin, or a nucleic acid vector encoding such a construct.

Cancers, including, but not limited to, neoplasms, tumors, metastases, or any disease or disorder characterized by angiogenesis-dependent uncontrolled cell growth, can be treated or prevented by administration of a construct of the invention, which construct specifically binds one or more binding partners related to the cancer to be treated or prevented. Whether a particular therapeutic is effective to treat or prevent a certain type of cancer can be determined by any method known in the art, for example but not limited to, the methods described in infra.

In other embodiments of the invention, the subject being treated with the construct may, optionally, be treated with other cancer treatments such as surgery, radiation therapy, or chemotherapy. In particular, the therapeutic of the invention used to treat or prevent cancer may be administered in conjunction with one or a combination of chemotherapeutic agents including, but not limited to, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc.

Malignancies and related disorders that can be treated or prevented by administration of a therapeutic of the invention include, but are not limited to, those listed in Table 1 (for a review of such disorders, see Fishman et al., Medicine 1985, 2d Ed., J. B. Lippincott Co., Philadelphia):

TABLE 1
SOLID TUMOR MALIGNANCIES AND RELATED DISORDERS

sarcomas and carcinomas
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon carcinoma
pancreatic cancer
breast cancer
ovarian cancer
prostate cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
lung carcinoma
small cell lung carcinoma
bladder carcinoma
epithelial carcinoma
glioma
astrocytoma
medulloblastoma
craniopharyngioma
ependymoma
pinealoma
hemangioblastoma
acoustic neuroma
oligodendroglioma
meningioma
melanoma
neuroblastoma
retinoblastoma

In specific embodiments, malignancy or dysproliferative changes (such as metaplasias and dysplasias), or hyperproliferative disorders, are treated or prevented in the ovary, bladder, breast, colon, lung, skin, pancreas, prostate, uterus, gastrointestinal tract. B lymphocytes or T lymphocytes.

### Gene Therapy

In a specific embodiment, vectors comprising a sequence encoding a construct of the invention are administered to treat or prevent a disease or disorder associated with inappropriate angiogenesis.

In this embodiment of the invention, the therapeutic vector encodes a sequence that produces intracellularly (without a signal sequence) or extracellularly (with a signal sequence) a construct of the invention. Preferably, the construct is secreted (produced extracellularly).

Any of the methods for gene therapy available in the art can be used according to the present invention. Exemplary methods are described below.

For general reviews of the methods of gene therapy, see, Goldspiel et al. Clinical Pharmacy, 1993, 12:488-505; Wu and Wu, Biotherapy, 1991, 3:87-95; Tolstoshev, Ann. Rev. Pharmacol. Toxicol., 1993, 32:573-596; Mulligan, Science, 1993, 260:926-932, and Morgan and Anderson, Ann. Rev. Biochem., 1993, 62:191-217; May, TIBTECH, 1993, 11:155-215. Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics. John Wiley & Sons, NY. Vectors suitable for gene therapy are described above.

In one aspect, the therapeutic vector comprises a nucleic acid that expresses the construct in a suitable host. In particular, such a vector has a promoter operationally linked to the coding sequence for the construct. The promoter can be inducible or constitutive and, optionally, tissue-specific. In another embodiment, a nucleic acid molecule is used in which the coding sequences and any other desired sequences are flanked by regions that promote homologous recombination at a desired site in the genome, thus providing for expression of the construct from a nucleic acid molecule that has integrated into the genome (Koller and Smithies, Proc. Natl. Acad. Sci. USA, 1989, 86:8932-8935; Zijlstra et al., Nature, 1989, 342:435-438).

Delivery of the vector into a patient may be either direct, in which case the patient is directly exposed to the vector or a delivery complex, or indirect, in which case, cells are first transformed with the vector in vitro, then transplanted into the patient. These two approaches are known, respectively, as in vivo and ex vivo gene therapy.

In a specific embodiment, the vector is directly administered in vivo, where it enters the cells of the organism and mediates expression of the construct. This can be accomplished by any of numerous methods known in the art and discussed above, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly-β-1-4-N-acetylglucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem., 1987, 62:4429-4432), etc. In another embodiment, a nucleic acid ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g. PCT Publication Nos. WO 92/06180. WO 92/22635, WO 92/20316 and WO 93/14188). These methods are in addition to those discussed above in conjunction with “Viral and Non-viral Vectors”.

Alternatively, single chain antibody constructs can also be administered, for example, by expressing nucleotide sequences encoding single-chain antibodies within the target cell population by utilizing, for example, techniques such as those described in Marasco et al. (Proc. Natl. Acad Sci. USA, 1993, 90:7889-7893).

The form and amount of therapeutic nucleic acid envisioned for use depends on the type of disease and the severity of the desired effect, patient state, etc., and can be determined by one skilled in the art.

### Formulations and Administration

Therapeutic compositions containing a construct for use in accordance with the present invention can be formulated in any conventional manner using one or more physiologically acceptable carriers or excipients.

Thus, the constructs or nucleic acids encoding them and their physiologically acceptable salts and solvents can be formulated for administration by inhalation (pulmonary) or insufflation (either through the mouth or the nose), by transdermal delivery, or by transmucosal administration, including, but not limited to, oral, buccal, nasal, opthalmic, vaginal, or rectal administration.

For oral administration, the therapeutics can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g. sodium lauryl sulphate). The tablets can be coated by methods well known in the art. Liquid preparations for oral administration can take the form of, for example, solutions, syrups, emulsions or suspensions, or they can be presented as a dry product for constitution with water or other suitable vehicle before use. Such liquid preparations can be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the therapeutics can take the form of tablets or lozenges formulated in conventional manner.

For administration by inhalation, the therapeutics according to the present invention are conveniently delivered in the form of an aerosol spray presentation from pressurized packs or a nebulizer with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the construct and a suitable powder base such as lactose or starch.

The therapeutics can be formulated for parenteral administration (e.g., intravenous, intramuscular, subcutaneous, intraderrnal) by injection, via, for example, bolus injection or continuous infusion. Formulations for injection can be presented in unit dosage form, e.g., in vials or ampoules, or in multi-dose containers, with an added preservative. The compositions can take such forms as excipients, suspensions, solutions or emulsions in oily or aqueous vehicles and can contain formulatory agents such as suspending, stabilizing and/or dispersing agents. Alternatively, the active ingredient can be in dry, lyophilized (i.e. freeze dried) powder form for constitution with a suitable vehicle, e.g., sterile pyrogen-free water or saline, before use.

The therapeutics can also be formulated in rectal compositions such as suppositories or retention enemas, e.g., containing conventional suppository bases such as cocoa butter or other glycerides.

In addition to the formulations described previously, the therapeutics can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In a specific embodiment, the constructs can be delivered in poly-glycolic acid/lactic acid (PGLA) microspheres (see U.S. Pat. Nos. 5,100,669 and 4,849,222; PCT Publication Nos. WO 95/11010 and WO 93/07861).

The constructs of the invention may be administered as separate compositions or as a single composition with more than one construct linked by conventional chemical or by molecular biological methods, e.g., by covalent attachment to polyethylene glycol (PEGylation). Additionally, the diagnostic and therapeutic value of the antibodies of the invention may be augmented by their use in combination with radionuclides or with toxins such as ricin or with chemotherapeutic agents such as methotrexate.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water-free concentrate in a sealed container such as a vial or sachette indicating the quantity of active agent. Where the composition is administered by injection, an ampoule of sterile diluent can be provided so that the ingredients may be mixed prior to administration.

The invention also provides a pharmaceutical pack or kit comprising one or more containers filled with one or more of the ingredients of the formulations of the invention. Associated with such container(s) can be a notice in the form prescribed by a governmental agency regulating the manufacture, use or sale of pharmaceuticals or biological products, which notice reflects approval by the agency of manufacture, use or sale for human administration.

The compositions may if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Composition comprising a compound of the invention formulated in a compatible pharmaceutical carrier may also be prepared, placed in an appropriate container, and labelled for treatment of an indicated condition.

Many methods may be used to introduce the vaccine formulations of the invention; these include but are not limited to oral, intracerebral, intradermal, intramuscular, intraperitoneal, intravenous, subcutaneous, intranasal routes, and via scarification (scratching through the top layers of skin, e.g., using a bifurcated needle) or any other standard routes of immunization.

### Vaccine Formulations and Administration

The invention also provides vaccine formulations containing therapeutics of the invention, which vaccine formulations are suitable for administration to elicit a protective immune (humoral and/or cell mediated) response against certain antigens, e.g. for the treatment and prevention of diseases.

Suitable preparations of such vaccines include injectables, either as liquid solutions or suspensions; solid forms suitable for solution in, suspension in, liquid prior to injection, may also be prepared. The preparation may also be emulsified, or the constructs antibodies encapsulated in liposomes. The active immunogenic ingredients are often mixed with excipients which are pharmaceutically acceptable and compatible with the active ingredient. Suitable excipients are, for example, water, saline, buffered saline, dextrose, glycerol, ethanol, sterile isotonic aqueous buffer or the like and combinations thereof. In addition, if desired, the vaccine preparation may also include minor amounts of auxiliary substances such as wetting or emulsifying agents, pH buffering agents, and/or adjuvants that enhance the effectiveness of the vaccine. The construct when prepared as a vaccine can be introduced in microspheres or microcapsules, e.g., prepared from PGLA (see, U.S. Pat. No. 5,814,344).

The effectiveness of an adjuvant may be determined by measuring the induction of anti-idiotype antibodies directed against the injected construct formulated with the particular adjuvant.

The composition can be a liquid solution, suspension, emulsion, tablet, pill, capsule, sustained release formulation, or powder. Oral formulations can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water-free concentrate in a sealed container such as a vial or sachette indicating the quantity of active agent. Where the composition is administered by injection, an ampoule of sterile diluent can be provided so that the ingredients may be mixed prior to administration.

In a specific embodiment, the lyophilized construct of the invention is provided in a first container; a second container comprises diluent consisting of an aqueous solution of 50% glycerin, 0.25% phenol, and an antiseptic (e.g., 0.005% brilliant green).

The invention also provides a pharmaceutical pack or kit comprising one or more containers filled with one or more of the ingredients of the vaccine formulations of the invention. Associated with such container(s) can be a notice in the form prescribed by a governmental agency regulating the manufacture, use or sale of pharmaceuticals or biological products, which notice reflects approval by the agency of manufacture, use or sale for human administration.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. Composition comprising a compound of the invention formulated in a compatible pharmaceutical carrier may also be prepared, placed in an appropriate container, and labeled for treatment of an indicated condition.

Many methods may be used to introduce the vaccine formulations of the invention; these include but are not limited to oral, intracerebral, intradermal, intramuscular, intraperitoneal, intravenous, subcutaneous, intranasal routes, and via scarification (scratching through the top layers of skin, e.g., using a bifurcated needle) or any other standard routes of immunization.

### Effective Dose

The constructs (both proteins and vectors encoding such proteins) described herein can be administered to a patient at therapeutically effective doses to treat certain diseases or disorders such as cancers. A therapeutically effective dose refers to that amount of a therapeutic sufficient to result in a healthful benefit in the treated subject.

The precise dose of the construct to be employed in the formulation will depend on the route of administration, and the nature of the patient's disease, and should be decided according to the judgment of the practitioner and each patient's circumstances according to standard clinical techniques. An effective immunizing amount (immunogenic amount) is that amount sufficient to produce an immune response to the constructs in the host (i.e., an anti-idiotype reaction) to which the vaccine preparation is administered. In either case, an effective dose is an amount effective to result in inhibition of angiogenin binding to actin in vivo. The term “inhibit” or “inhibition” means to reduce by a measurable or observable amount. The ability of a therapeutic composition or vaccine of the invention to produce this effect can be detected in vitro, e.g., using a competitive binding assay with labeled angiogenin as exemplified infra. Such an assay can be formatted in a solid phase format, in which actin or angiogenin is adsorbed to a solid support or in a cellular format. Further experimental evidence of inhibition includes observing inhibition of angiogenesis in vitro or in vivo, and inhibition of tumor growth or metastasis, or both, in an animal model. The degree of inhibition is at least sufficient for measurement; preferably, it is at least about 5%; more preferably from about 5% to about 50%; more preferably still greater than about 50%; and most preferably greater than about 95%. Effective doses may be extrapolated from dose-response curves derived from animal model test systems.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutics that exhibit large therapeutic indices are preferred. While therapeutics that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀) (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

### EXAMPLES

The following Examples illustrate the invention without limiting it.

### Example 1

### Construction of the Variable Region Gene Containing the Angiogenin Binding Site

The variable region gene encoding a CDR containing the actin-binding site from angiogenin is constructed with the following cloning steps using standard conditions. First, a “PCR knitting” protocol was developed (see example diagramed in FIG. 2) to replace existing CDR sequences with the desired sequences from angiogenin. In this protocol, positive and negative strand oligonucleotide primers were designed that overlap approximately ten residues at the 5′ end and contain novel sequences at their 5′ ends that encode the angiogenin peptide sequences. At the 3′ ends, the oligonucleotides contain sequences homologous to the framework sequences adjacent to the CDR being modified. Two polymerase chain reactions (PCR) were then performed using as primer pairs, one primer that encodes a portion of the peptide sequence and an appropriate primer up- or downstream from the CDR to be modified that corresponds to framework sequences that flank the CDR (FIG. 2). The template DNA used for these two PCR reactions was the consensus variable region that has been described previously (FIGS. 1A and 1B), which was cloned into the shuttle vector pUC19 using standard techniques. PCR reactions were initiated by incubating the reactions for 10 minutes at 95° C. and then running 25 cycles of 30 seconds at 95° C., followed by 30 seconds at 55° C. and followed by 30 seconds at 72° C. After 25 cycles, an additional incubation is performed for seven minutes at 72° C. The two PCR reactions produce DNA fragments that overlap by approximately ten base pairs at one of their termini (FIG. 2; Table 2) and these fragments were purified using QIAquick PCR purification columns according to the manufacturer's instructions (Qiagen).

The isolated fragments were then “knitted” together in another PCR reaction in which the flanking primers used in the first two PCR reactions described above were included in the reaction along with the two DNA fragments. PCR reactions were begun by incubating for 10 minutes at 95° C., then 5 cycles were run of 30 seconds at 94° C., followed by 1 minute at 40° C. and followed by 30 seconds at 72° C. Twenty additional cycles were then performed of 30 seconds at 94° C., followed by 30 seconds at 55° C., and followed by 30 seconds at 72° C. After 25 cycles, an additional incubation is performed for seven minutes at 72° C. The product of this reaction was a longer DNA fragment that results from the joining of the initial two fragments by selective annealing of the DNA fragments through the overlapping sequences present at one of their termini followed by amplification with the flanking primers (FIG. 2).

TABLE 2

Sequences of primers used for preparation of angiogenin constructs.
Sequences are shown in 5′ to 3′ orientation.

| | |
|---|---|
| Primer (SEQ ID NO:) | Sequence |

| | |
|---|---|
| ANGIOVHP2 (5) | TGTGAGGGTTTCCATTCTTGTTTTCTGTGAATGTGTAGCCAGAAGC |
| ANGIOVHP3 (6) | AACCCTCACAGAGAAAACCTAAGATGGGTGAGGCAGGCTCCC |
| ANGIOVHP4 (7) | TGTGAGGGTTTCCATTCTTGTTTTCGCCCATCCACTCCAGGCC |
| ANGIOVHP5 (8) | AACCCTCACAGAGAAAACCTAAGAAGGGTTACTATAACTGCTGATAC |
| ANGIOVHF6 (9) | TGTGAGGGTTTCCATTCTTGTTTTCCCTAGCGCAGTAGTAAACAG |
| ANGIOVHP7 (10) | AACCCTCACAGAGAAAACCTAAGATGGGGACAGGGAACACTGG |
| ANGIOVLP2 (11) | TGTGAGGGTTTCCATTCTTGTTTTCACATGTGATTGTCACCCGATC |
| ANGIOVLP3 (12) | AACCCTCACAGAGAAAACCTAAGATGGTATCAACAAAAGCCC |
| ANGIOVLP4 (13) | TGTGAGGGTTTCCATTCTTGTTTTCATAGATCAACAACTTAGGAGCC |
| ANGIOVLP5 (14) | AACCCTCACAGAGAAAACCTAAGAGGAGTGCCTAGTCGGTTC |
| ANGIOVLP6 (15) | TGTGAGGGTTTCCATTCTTGTTTTCACAATAATAGGTAGCGAAGTC |
| ANGIOVLP7 (16) | AACCCTCACAGAGAAAACCTAAGATTCGGACAAGGAACCAAGGTG |
| VHLP1-Forward -20 (17) | GTAAAACGACGGCGAGT |
| VHLP8-Reverse -48 (18) | AGCGGATAACAATTTCACACAGGA |
| VLP3(19) | AGATCGGGTGACAATCACATG |
| VLP4 (20) | CGTGTTCCACTTCCACTTCC |
| VLP5 (21) | GGAGTGCCTAGTCGGTTC |
| VLP6 (22) | CACCTTGGTTCCTTGTCCG |
| VHP3 (23) | GTCTTGCAAGGCTTCTGGC |
| VHP4 (24) | ATCAGCAGTTATAGTAACCCT |
| VHP5 (25) | AGGGTTACTATAACTGCTGAT |
| VHP6 (26) | CCTAGCGCAGTAGTAAACAG |

To facilitate the cloning of the modified CDR containing the angiogenin sequences back into the consensus variable region clone, unique restriction sites were inserted into the flanking sequences, one on either side of each CDR (FIG. 2), using the QuikChange™ kit from Stratagene according to the manufacturer's instructions. The “knitted” PCR fragment was then cleaved with the appropriate restriction enzymes (FIG. 2) and ligated into the cloned consensus variable region that had been cut with the same restriction enzymes.

The assembled, modified variable region containing the angiogenin sequences was then linked to the appropriate constant region clone. For assembly of the heavy chain of the antibody, a unique XhoI restriction enzyme site was engineered into both the 3′ end of the variable region and the 5′ end of the IgG1 heavy chain constant region. At the 5′ end of the variable region, an EcoRI restriction site and a Kozak sequence were added using PCR. The modified heavy chain variable region was then joined to the heavy chain constant region by inserting the EcoRI/XhoI cut variable region fragment into a vector obtained from Lonza Biologics PLC, containing the EcoRI/XhoI cut heavy chain constant region. For assembly of the light chain of the antibody, a unique Bg/II restriction enzyme site was engineered into the 3′ end of the light chain variable region and a BclI restriction enzyme site was added to the 5′ end of the light chain constant region (K chain). Similar to the heavy chain variable region, an EcoRI restriction site and Kozak sequence were added to the 5′ end of the light chain variable region using PCR. When BglII and BclI cut their respective cleavage sites, both enzymes leave overhangs with the same DNA sequence, which allows them to be ligated. Consequently, the modified light chain variable region clone was digested with EcoRI/BglII and the resulting fragment inserted into a second vector, obtained from Lonza Biologics PLC, containing the EcoRI/BclI cut light chain constant region.

In a final step, the heavy chain expression vector, containing the heavy chain variable region, and the light chain expression vector, containing the light chain variable region, were assembled into a single “double gene” expression vector. To assemble the “double gene” vector, the heavy chain expression vector was cleaved with BamHI and NotI. The resulting fragment contains the complete heavy chain expression cassette including the CMV promoter, the assembled heavy chain and a transcriptional terminator. The light chain expression vector was also cleaved with BamHI and NotI and after purifying the vector from a small fragment, the heavy chain expression cassette is inserted into the light chain vector.

Peptide sequences of variable regions containing angiogenin binding sequences. Inserted angiogenin sequences in each construct are indicated by underlining. Peptide sequences of consensus heavy chain (CONVH) and consensus light chain (CONUVL) variable regions are also shown, with CDRs underlined.

| | | |
|---|---|---|
| ANGVHCDR1 | MetAlaTrpValTrpThrLeuLeuPheLeuMetAlaAlaAlaGlnSerAlaGlnAla | (SEQ ID NO:27) |
| | GlnValGlnLeuValGlnSerGlyAlaGluValLysLysProGlyAlaSerValLyVal | |
| | Ser | |
| | CysLysAlaSerGlyTyrThrPheThrGluAsnLysAsnGlyAsnProHisArgGlu | |
| | AsnLeuArgTrpValArgGlnAlaProGlyGlnGlyLeuGluTrpMetGlyTrpIle | |
| | AsnGly | |
| | AsnGlyAspThrAsnTyrAlaGlnLysPheGlnGlyArgValThrIleThrAlaAsp | |
| | Thr | |
| | SerThrSerThrAlaTyrMetGluLeuSerSerLeuArgSerGluAspThrAlaVal | |
| | Tyr | |
| | TyrCysAlaArgAlaProGlyTyrGlySerAspTyrTrpGlyGlnGlyThrLeuVal | |
| | ThrValSerSer | |
| ANGVHCDR2 | MetAlaTrpValTrpThrLeuLeuPheLeuMetAlaAlaAlaGlnSerAlaGlnAla | (SEQ ID NO:28) |
| | GlnValGlnLeuValGlnSerGlyAlaGluValLysLysProGlyAlaSerValLys | |
| | ValSer | |
| | CysLysAlaSerGlyTyrThrPheThrSerTyrAlaIleSerTrpAsnTrpValArgGln | |
| | AlaProGlyGlnGlyLeuGluTrpMetGlyGluAsnLysAsnGlyAsnProHisArg | |
| | GluAsnLeuArgArgValThrIleThrAlaAspThrSerThrSerThrAlaTyrMet | |
| | Glu | |
| | LeuSerSerLeuArgSerGluAspThrAlaValTyrTyrCysAlaArgAlaProGly | |
| | Tyr GlySerAspTyrTrpGlyGlnGlyThrLeuValThrValSerSer | |
| ANGVHCDR3 | MetAlaTrpValTrpThrLeuLeuPheLeuMetAlaAlaAlaGlnSerAlaGlnAla | (SEQ ID NO:29) |
| | GlnValGlnLeuValGlnSerGlyAlaGluValLysLysProGlyAlaSerValLys | |
| | ValSer | |
| | CysLysAlaSerGlyTyrThrPheThrSerTyrAlaIleSerTrpAsnTrpValArgGln | |
| | AlaProGlyGlnGlyLeuGluTrpMetGlyTrpIleAsnGlyAsnGlyAspThrAsn | |
| | TyrAlaGlnLysPheGlnGlyArgValThrIleThrAlaAspThrSerThrSerThr | |
| | AlaTyr | |
| | MetGluLeuSerSerLeuArgSerGluAspThrAlaValTyrTyrCysAlaArgGlu | |
| | AsnLysAsnGlyAsnProHisArgGluAsnLeuArgTrpGlyGlnGlyThrLeuVal | |
| | Thr ValSerSer | |
| ANGVLCDR1 | MetAlaTrpValTrpThrLeuLeuPheLeuMetAlaAlaAlaGlnSerAlaGlnAla | (SEQ ID NO:30) |
| | Asp | |
| | IleGlnMetThrGlnSerProSerSerLeuSerAlaSerValGlyAspArgValThrIle | |
| | Thr | |
| | CysGluAsnLysAsnGlyAsnProHisArgGluAsnLeuArgTrpTyrGlnGln | |
| | Lys | |
| | ProGlyLysAlaProLysLeuLeuIleTyrAlaAlaSerSerLeuGluSerGlyValPro | |
| | SerArgPheSerGlySerGlySerGlyThrArgPheThrLeuThrIleSerSerLeuGln | |
| | Pro | |
| | GluAspPheAlaThrTyrTyrCysGlnGlnTyrAsnSerLeuProTrpThrPheGly | |
| | GlnGlyThrLysValGluIle Lys | |
| ANGVLCDR2 | MetAlaTrpValTrpThrLeuLeuPheLeuMetAlaAlaAlaGlnSerAlaGlnAla | (SEQ ID NO:31) |
| | Asp | |
| | IleGlnMetThrGlnSerProSerSerLeuSerAlaSerValGlyAspArgValThrIle | |
| | Thr | |
| | CysArgAlaSerGlnSerIleSerAsnTyrLeuAlaTrpTyrGlnGlnLysProGly | |
| | Lys | |
| | AlaProLysLeuLeuIleTyrGluAsnLysAsnGlyAsnProHisArgGluAsnLeu | |
| | ArgGlyValProSerArgPheSerGlySerGlySerGlyThrArgPheThrLeuThrIle | |
| | SerSerLeuGlnProGluAspPheAlaThrTyrTyrCysGlnGlnTyrAsnSerLeu | |
| | ProTrpThrPheGlyGlnGlyThrLysValGluIleLys | |
| ANGVLCDR3 | MetAlaTrpValTrpThrLeuLeuPheLeuMetAlaAlaAlaGlnSerAlaGlnAla | (SEQ ID NO:32) |
| | Asp | |
| | IleGlnMetThrGlnSerProSerSerLeuSerAlaSerValGlyAspArgValThrIle | |
| | Thr | |
| | CysArgAlaSerGlnSerIleSerAsnTyrLeuAlaTrpTyrGlnGlnLysProGly | |
| | Lys | |
| | AlaProLysLeuLeuIleTyrAlaAlaSerSerLeuGluSerGlyValProSerArgPhe | |
| | SerGlySerGlySerGlyThrArgPheThrLeuThrIleSerSerLeuGlnProGluAsp | |
| | Phe | |
| | AlaThrTyrTyrCysGluAsnLysAsnGlyAsnProHisArgGluAsnLeuArgPhe | |
| | GlyGlnGlyThrLysValGluIleLys | |

The nucleotide sequences of the VH and VL consensus sequences are also provided:

VHCON

(SEQ ID NO:33)

ATGGCTTGGGTGTGGACCTTGCTATTCCTGATGGCAGCTGCCCAAAGTGC
CCAAGCACAGGTTCAGCTGGTGCAGTCTGGCGCTGAGGTGAAGAAGCCTG
GCGCTTCTGTGAAGGTGTCTTGCAAGGCTTCTGGCTACACATTCACATCT
TACGCTATATCTTGGAATTGGGTGAGGCAGGCTCCCGGGCAGGGCCTGGA
GTGGATGGGCTGGATAAATGGAAATGGAGATACAAATTACGCCCAGAAGT
TCCAGGGAAGGGTTACTATAACTGCTGATACTTCTACTTCTACTGCTTAC
ATGGAGCTCTCTTCTCTGAGGTCTGAGGATACTGCTGTTTACTACTGCGC
TAGGGCTCCTGGCTACGGCTCTGATTATTGGGGACAGGGAACACTGGTTA
CAGTCTCGAGT
VLCON

(SEQ ID NO:34)

ATGGCTTGGGTGTGGACCTTGCTATTCCTGATGGCAGCTGCCCAAAGTGC
CCAAGCAGATATCCAAATGACACAAAGTCCTAGTAGTTTGAGTGCTAGTG
TGGGAGATCGGGTGACAATCACATGTCGGGCTAGTCAAAGTATCAGTAAC
TATTTGGCTTGGTATCAACAAAAGCCCGGGAAGGCTCCTAAGTTGTTGAT
CTATGCTGCTAGTAGTTTGGAGAGTGGAGTGCCTAGTCGGTTCAGTGGAA
GTGGAAGTGGAACACGGTTCACCTTGACCATCAGTAGTTTGCAACCTGAA
GACTTCGCTACCTATTATTGTCAACAATATAACAGTTTGCCTTGGACCTT
CGGACAAGGAACCAAGGTGGAGATCAAG

### Example 2

### Protein Expression

Once constructs were prepared, initial transfections were performed transiently in CHO-K1 cells. Cotransfections were performed using two single gene constructs and a cationic liposomal reagent. Expression was measured at day 3 and day 7 by ELISA assay. The expressed synthetic antibody was purified using Protein-A or Protein-G column chromatography and characterized by HPLC and Western immunoblotting.

Stable transfectants can be produced in a number of cell lines including but not limited to CHO-K1, NSO and HEK-293. The choice of which cell line to use depends on a number of factors, some of which include: glycosylation patterns, expression level and ability to adapt to serum-free or protein-free media.

### Example 3

### Assessment of Angiogenin Synthebody Activity—Binding to Actin

Due to the steric structure of the antibody, the heterologous binding sequence inserted into different CDRs may bind to actin with different affinity. An ELISA assay was developed to examine the binding of various synthebody constructs to actin.

### Experimental Procedure

Bicarbonate coating buffer (Sigma # C-3041) containing 5 μg/ml bovine actin (Sigma # A-3653) was added to an Immunlon IV microtiter plate (VWR # 62402-959) at 100 μl/well. After an incubation at 4° C. overnight, the coating buffer was removed and the plate was washed 3 times with wash buffer (PBS+0.1% Tween-20). The plate was then blocked with 1% BSA in PBS-T for 2 hours at room temperature followed by 3 washes with wash buffer for 3 minutes each. Synthebodies were diluted with fresh ProCho4 CDM medium (BioWhittaker # 12-029-Q) to 1 μg/ml for the binding assay and serially 1:1 for the dose response assay (synthebody concentrations were 20, 10, 5, 2.5, 1.25, 0.625, 0.313, and 0.16 μg/ml, corresonding to approximately 133, 66, 33, 16.7, 8.3, 4.16, 2.08, and 1.04 nM, respectively; control antibody was used at the same concentrations except for the lowest), and were added to the plate at 100 μl/well in triplicate wells. Following incubation at room temperature for 2 hours, the wells were washed 3 times in wash buffer. Goat anti-human IgG-HRP conjugate (Southern Biotechnology #2040-05) was diluted 1:1000 in 1% BSA-PBS and added to the plate at 100 μl/well. Following incubation for 1 hour at room temperature, the wells were again washed 3 times with wash buffer. The substrate solution was prepared by mixing equal volume of H₂O₂ and TMB (KPL # 50-76-00) immediately before use and 100 μl/well were added to the plate. The color development was measured as optical densitv at a wavelength of 650 using an automated microplate reader (Molecular Devices).

### Results

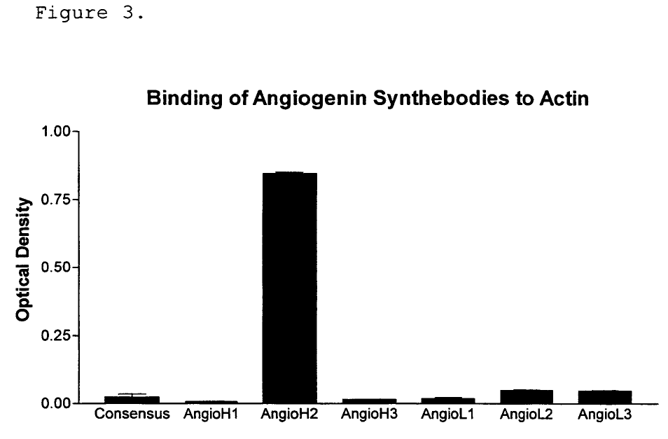

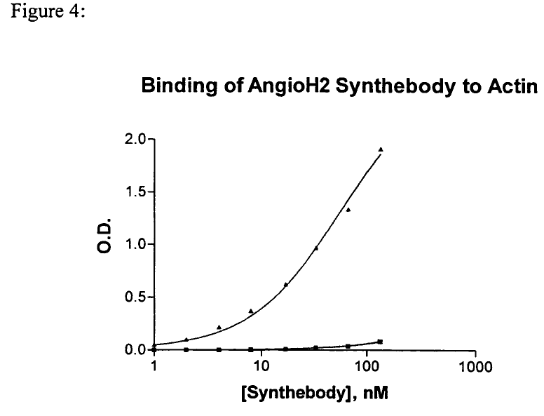

In this assay, various synthebody preparations were added to an actin coated Immulon IV plate (5 mg/ml). Binding was detected by using goat anti-human-HRP conjugate plus substrate. One of the synthebodies (AngioH2) demonstrated the ability to bind to actin (FIG. 3). As shown in FIG. 4, the AngioH2 synthebody showed dose dependent binding of actin. The K_{d} of the synthebodv was determined using saturating doses of the synthebody in this assay.

### Example 4

### Assessment of Angiogenin Synthebody Activit—Blocking the Binding of Angiogenin to Actin

The ability of the synthebody to block the interaction between angiogenin and actin was evaluated with an ELISA competition assay.

### Experimental Procedure

The Immunlon IV microtiter plate with serially diluted (1:1) AngioH2 antibody was prepared as described in Example 3. Concentrations of AngioH2 synthebody (and control consensus synthebody) were 6, 3, 1.5, 0.75, and 0.375 μg/ml (40, 20, 10, 5, and 2.5 nM, respectively). Bicarbonate coating buffer (Sigma # C-3041) containing 5 mg/ml bovine actin (Sigma # A-3653) was added to an Immunlon IV microtiter plate (VWR # 62402-959) at 100 ml/well. Recombinant human (rh) angiogenin (R & D Systems # MAB265) at 5 μg/ml was added to the synthebodies at 10 μl/well, so the final concentration of rh-angiogenin in each well was 0.5 μg/ml (36 nM). Following incubation at room temperature for 2 hours, the wells were washed 3 times with wash buffer. Monoclonal anti-angiogenin (R&D Systems #MAB265, diluted 1:250) was mixed with goat anti-mouse IgG-HRP conjugate (Southern Biotechnology 1070-05, diluted 1:1000 in 1% BSA-PBS) and added to the plate at 100 μl/well. Following incubation for 1 hour, the wells were again washed 3 times with wash buffer. The substrate solution and color reaction were prepared and monitored as described in Example 3. The angiogenin bound to actin was quantified by comparing color development for the sample to a standard curve performed on the same plate.

### Results

In this assay, actin was coated on an Immulon IV plate as described for Example 3. A constant concentration of recombinant angiogenin (36 nM) was competed by various concentrations of angiogenin synthebodies. The bound angiogenin was detected by using anti-angiogenin-HRP plus substrate. The results demonstrate that AngioH2 blocks the binding of recombinant human angiogenin to actin with an approximate Kᵢ of 4.2 nM.

### Example 5

### Assessment of Angiogenin Synthebody Activity on Cellular Function In Vitro and In Vivo

Binding to endothtelial cells. A relevant target for a successful synthebody appears to be the endothelial cell. Thus, binding of angiogenin and angiogenin synthebodies is assessed using cultured vascular endothelial cells. Pooled human umbilical vein endothelial cells (HUVEC P157) are obtained at passage one from Clonetics and are maintained in Endothelial Cell Growth Medium-2 (EGM-2) with 2% Fetal Calf Serum (FCS) plus additional growth factors: EGF, bFGF. VEGF and IGF-1. For flow cytometry analysis, HUVEC cells (passage 4) are harvested from the culture by trypsinization and are washed twice with FACS buffer (1% BSA in PBS). The cells are seeded in a 96-well round bottom plate at 1×10⁶ cells/well. Excess buffer is removed by centrifugation of the plate for 3 minutes at 1200 rpm followed by gentle aspiration. Recombinant human angiogenin at various concentrations (250, 50, 10, 2 and 0.4 μg/ml) is added to the wells at 50 μl/well. After 30 minutes incubation at 4° C., the cells are washed 3 times with FACS buffer and are incubated with 50 μl of monoclonal anti-human angiogenin antibody (R & D System) at 20 μg/ml for 30 minutes at 4° C. The cells are then washed 3 times again with FACS buffer and are incubated with 50 μl/well of FITC-conjugated goat anti-mouse IgG (Southern Technology Associates) for 30 minutes at 4° C. Finally, the cells are washed 3 times with FACS buffer, are transferred to Falcon 2052 tubes, are resuspended in 400 μl FACS buffer, and are analyzed on the flow cytometer. A dose response curve is plotted as a percentage of positive-binding cells.

Inhibition of angiogenesis. The effect of the synthebody on angiogenesis is tested in rat aorta vessel outgrowth assays (Nicossia and Ottinetti, Lab Invest. 1990, 63:115-22).

In this assay, thoracic aorta are obtained under sterile conditions from 1-2 month old Fischer 344 male rats that are sacrificed by CO₂ asphyxiation. After exposing the posterior mediastinum, the aorta is ligated with silk sutures (3-0) proximally, below the aortic arch, and distally, above the diaphragm. The aorta is then dissected away from the vertebral column, is excised below the proximal suture, and is transferred to a compartmentalized Felsen dish containing serum-free MCDB 131-growth medium. Each rat aorta is cut into 18-20 rings (by cross-section) ranging from 1-2 mm in length and is embedded in a collagen or fibrin gel emersed in medium. The outgrowth of new vessels is enumerated visually every 2-3 days from day 3 to day 14. Dose dependent vessel growth curves are established with recombinant human angiogenin. Synthebodies may be tested at different concentrations in the presence or absence of recombinant human angiogenin. The growth curves of the vessels are compared, and a successful synthebody construct demonstrates activity in both assays.

Suppression of tumor growth. The efficacy of the synthebody in inhibiting tumor growth is evaluated using human tumor xenograft models in nude mice. In these experiments, one to three million human colon carcinoma cells (LS 174T or HT-29) are administered to groups of nude mice (5 to 8 animals per group) by subcutaneous injection in the left flank to establish the xenografts. Various doses of angiogenin synthebody (ranging from 10-200 μg) are then injected into the mice (i.v. or i.p.) at different times during tumor development (day 0, 3, 7 or 10). The size of the tumor is measured three times a week using calipers and is compared with the different experimental groups. An effective synthebody inhibits tumor growth relative to untreated or mock treated controls, preferably in a dose-dependent fashion. Ideally, the synthebody can cause tumor regression.

### Example 6

### Anti-Idiotype Synthebody

Anti-idiotype inducing constructs (in which the disulfide bonds in the variable region are disrupted) are used to immunize Balb/C mice. Five to eight mice are used in each treatment group, and the animals first receive a priming injection of 100 μg of an anti-idiotype inducing or control antibody suspended in Complete Freund's Adjuvant. The mice then receive three boosts of 50 μg of the synthetic antibodies suspended in Incomplete Freund's Adjuvant once every three weeks.

Animals are bled once every three weeks, and their sera are tested for anti-idiotype antibody levels by ELISA. If the anti-idiotypic antibody levels reached are 1 μg/ml, the animals may be challenged with 5×10⁴ CT-26 syngeneic colon carcinoma cells in the flank region by subcutaneous injection. The animals are examined for the formation of a tumor as well as the size of any tumor three times a week for two weeks. The animals that are not immunized or are mock immunized only with adjuvant serve as controls.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated by reference in their entirety for all purposes.

What is claimed is:

1. A variant of an immunoglobulin variable domain, said immunoglobulin variable domain comprising (A) at least one CDR region and (B) framework regions flanking said CDR, said variant comprising:
(a) said CDR region having added or substituted therein at least one binding sequence and
(b) said flanking framework regions, wherein said binding sequence is heterologous to said CDR and is a binding sequence from a binding site of a binding pair, and wherein said binding sequence is a receptor-binding portion of angiogenin.

2. A variant as defined in claim 1, wherein (i) one or more amino acid residues in one or more of said flanking framework regions has been substituted or deleted, (ii) one or more amino acid residues has been added in one or more of said flanking framework regions, or (iii) a combination of (i) and (ii).

3. A variant as defined in claim 1, wherein (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

4. A variant as defined in claim 1, wherein (i) one or more amino acid residues in one or more of said flanking framework regions has been substituted or deleted, (ii) one or more amino acid residues has been added in one or more of said flanking framework regions, or (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

5. A variant of an immunoglobulin variable domain, said immunoglobulin variable domain comprising (A) at least one CDR region and (B) framework regions flanking said CDR, said variant comprising
(a) said CDR region having added or substituted therein at least one amino acid sequence which is heterologous to said CDR and
(b) said flanking framework regions,
wherein said heterologous sequence is capable of binding to a target sequence or molecule, and
wherein said heterologous sequence is a receptor-binding portion of angiogenin.

6. A variant as defined in claim 5, wherein (i) one or more amino acid residues in one or more of said flanking framework regions has been substituted or deleted, (ii) one or more amino acid residues has been added in on or more of said flanking framework regions, or (iii) a combination of (i) and (ii).

7. A variant as defined in claim 5, wherein (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

8. A variant as defined in claim 5, wherein (i) one or more amino acid residues in one or more of said flanking framework regions has been substituted or deleted, (ii) one or more amino acid residues has been added in one or more of said flanking framework regions, (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

9. A variant as defined in claim 5, wherein said receptor-binding portion of angiogenin has a sequence corresponding to about amino acid residue 58 to about amino acid residue 70 of angiogenin.

10 A variant as defined in claim 5, wherein said receptor binding portion of angiogenin has an amino acid sequence ENKNGNPHRENLR (SEQ ID NO:4).

11. A variant as defined in claim 5, wherein said receptor-binding portion of angiogenin is in more than one CDR.

12. A variant as defined in claim 5, wherein said receptor-binding portion of angiogenin is in a CDR of a heavy chain variable region.

13. A variant as defined in claim 5, wherein said heterologous sequence comprises the amino acid sequence ENKNGNPHRENLR (SEQ ID NO:4) in CDR2 of a human heavy chain variable region.

14. A variant as defined in claim 5, wherein said variable region contains one or more cysteine residues that form at least one intrachain disulfide bond and at least one of said disulfide bonds is disrupted.

15. A variant as defined in claim 14, wherein one or more of said disulfide bonds is disrupted by substitution with a non-disulfide-forming amino acid at a position corresponding to a position selected from the group consisting of residue 23 of the light chain variable region, residue 88 of the light chain variable region, residue 22 of the heavy chain variable region, and residue 92 of the heavy chain variable region or the Kabat equivalent.

16. A variant as defined in claim 5, wherein said heterologous sequence is capable of specifically binding to said target sequence or molecule.

17. A variant as defined in claim 5, wherein said CDR region is CDR 1.

18. A variant as defined in claim 5, wherein said CDR region is CDR 2.

19. A variant as defined in claim 5, wherein said CDR region is CDR 3.

20. A variant as defined in claim 5, which is an antibody.

21. A variant as defined in claim 5, wherein said target sequence or molecule is antigenic.

22. A molecule comprising a variant as defined in claim 5.

23. A molecule comprising a variant as defined in claim 6.

24. A molecule comprising a variant as defined in claim 7.

25. A molecule comprising a variant as defined in claim 8.

26. A molecule comprising a variant as defined in claim 9.

27. A molecule comprising a variant as defined in claim 10.

28. A molecule comprising a variant as defined in claim 13.

29. A molecule comprising a variant as defined in claim 14.

30. A molecule comprising a variant as defined in claim 15.

31. A molecule as defined in claim 22, further comprising one or more constant domains from an immunoglobulin.

32. A molecule as defined in claim 22, further comprising a second variable domain linked to said variant.

33. A molecule as defined in claim 22, further comprising a second variable domain linked to said variant, and one or more constant domains from an immunoglobulin.

34. A molecule as defined in claim 22, wherein said heterologous sequence is capable of specifically binding to said target sequence or molecule.

35. A molecule as defined in claim 22, wherein said CDR region is CDR 1.

36. A molecule as defined in claim 22, wherein said CDR region is CDR 2.

37. A molecule as defined in claim 22, wherein said CDR region is CDR 3.

38. A molecule as defined in claim 22, which is an antibody.

39. A molecule as defined in claim 22, wherein said target sequence or molecule is antigenic.

40. A molecule as defined in claim 22, which comprises an amino acid sequence as depicted in SEQ ID NO:28.

41. A molecule as defined in claim 22, which is derived from a human antibody.

42. A molecule as defined in claim 22, which is derived from a chimeric or a humanized antibody.

43. An immunoglobulin comprising a heavy chain and a light chain, wherein said heavy chain comprises a variant as defined in claim 5 and three constant domains from an immunoglobulin heavy chain, and said light chain comprises a second variable domain associated with said variant and a constant domain from an immunoglobulin light chain.

44. An immunoglobulin comprising a heavy chain and a light chain, wherein said light chain comprises a variant as defined in claim 5 and a constant domain from an immunoglobulin light chain, and said heavy chain comprises a second variable domain associated with said variant and three constant domains from an immunoglobulin heavy chain.

45. An isolated nucleic acid encoding a variant as defined in claim 1.

46. An isolated nucleic acid encoding a variant as defined in claim 5.

47. An isolated nucleic acid encoding a molecule as defined in claim 22.

48. An isolated nucleic acid encoding an immunoglobulin as defined in claim 43.

49. An isolated nucleic acid encoding an immunoglobulin as defined in claim 44.

50. A cell containing nucleic acid as defined in claim 45.

51. A cell containing nucleic acid as defined in claim 46.

52. A cell containing nucleic acid as defined in claim 46.

53. A cell containing nucleic acid as defined in claim 47.

54. A cell containing nucleic acid as defined in claim 101.

55. A recombinant non-human host containing nucleic acid as defined in claim 45.

56. A recombinant non-human host containing nucleic acid as defined in claim 46.

57. A recombinant non-human host containing nucleic acid as defined in claim 46.

58. A recombinant non-human host containing nucleic acid as defined in claim 47.

59. A recombinant non-human host containing nucleic acid as defined in claim 48.

60. A pharmaceutical composition comprising a therapeutically or prophylactically effective amount of a variant as defined in claim 1, and a pharmaceutically acceptable carrier.

61. A pharmaceutical composition comprising a therapeutically or prophylactically effective amount of a variant as defined in claim 5, and a pharmaceutically acceptable carrier.

62. A pharmaceutical composition comprising a therapeutically or prophylactically effective amount of a variant as defined in claim 9, and a pharmaceutically acceptable carrier.

63. A pharmaceutical composition comprising a therapeutically or prophylactically effective amount of a variant as defined in claim 10, and a pharmaceutically acceptable carrier.

64. A pharmaceutical composition comprising a therapeutically or prophylactically effective amount of a variant as defined in claim 13, and a pharmaceutically acceptable carrier.

65. A pharmaceutical composition comprising a therapeutically or prophylactically effective amount of a variant as defined in claim 14, and a pharmaceutically acceptable carrier.

66. A pharmaceutical composition comprising a therapeutically or prophylactically effective amount of a variant as defined in claim 15, and a pharmaceutically acceptable carrier.

67. A pharmaceutical composition comprising a therapeutically or prophylactically effective amount of a molecule as defined in claim 22, and a pharmaceutically acceptable carrier.

68. A pharmaceutical composition comprising a therapeutically or prophylactically effective amount of an immunoglobulin as defined in claim 43, and a pharmaceutically acceptable carrier.

69. A pharmaceutical composition comprising a therapeutically or prophylactically effective amount of an immunoglobulin as defined in claim 44, and a pharmaceutically acceptable carrier.

70. A vaccine comprising an effective amount of a variant as defined in claim 1 to induce an immune response.

71. A vaccine comprising an effective amount of a variant as defined in claim 5 to induce an immune response.

72. A vaccine comprising an effective amount of a molecule as defined in claim 22 to induce an immune response.

73. A vaccine comprising an effective amount of an immunoglobulin as defined in claim 58 to induce an immune response.

74. A vaccine comprising an effective amount of a variant as defined in claim 1 to induce an anti-idiotype response.

75. A vaccine comprising an effective amount of a variant as defined in claim 5 to induce an anti-idiotype response.

76. A vaccine comprising an effective amount of a molecule as defined in claim 22 to induce an anti-idiotype response.

77. A vaccine comprising an effective amount of an immunoglobulin as defined in claim 43 to induce an anti-idiotype response.

78. A vaccine comprising an effective amount of an immunoglobulin as defined in claim 44 to induce an anti-idiotype response.

79. A method of treating or preventing a disease in a subject in need of such treatment or prevention, said method comprising administering to said subject a disease treating or preventing effective amount of a variant as defined in claim 1, wherein (i) said disease is caused directly or indirectly by an agent, (ii) a symptom of said disease is caused by an agent, or (iii) said disease produces a physical, chemical, or biological response, wherein said agents or response include said target sequence or molecule.

80. A method of treating or preventing a disease in a subject in need of such treatment or prevention, said method comprising administering to said subject a disease treating or preventing effective amount of a variant as defined in claim 5, wherein (i) said disease is caused directly or indirectly by an agent, (ii) a symptom of said disease is caused by an agent, or (iii) said disease produces a physical, chemical, or biological response, wherein said agents or response include said target sequence or molecule.

81. A method of treating or preventing a disease in a subject in need of such treatment or prevention, said method comprising administering to said subject a disease treating or preventing effective amount of a molecule as defined in claim 22, wherein (i) said disease is caused directly or indirectly by an agent, (ii) a symptom of said disease is caused by an agent, or (iii) said disease produces a physical, chemical, or biological response, herein said agents or response include said target sequence or molecule.

82. A method of treating or preventing a disease in a subject in need of such treatment or prevention, said method comprising administering to said subject a disease treating or preventing effective amount of an immunoglobulin as defined in claim 43, wherein (i) said disease is caused directly or indirectly by an agent, (ii) a symptom of said disease is caused by an agent, or (iii) said disease produces a physical, chemical, or biological response, wherein said agents or response includes said target sequence or molecule.

83. A method of treating or preventing a disease in a subject in need of such treatment or prevention, said method comprising administering to said subject a disease treating or preventing effective amount of an immunoglobulin as defined in claim 44, wherein (i) said disease is caused directly or indirectly by an agent, (ii) a symptom of said disease is caused by an agent, or (iii) said disease produces a physical, chemical, or biological response, wherein said agents or response include said target sequence or molecule.

84. A method of treating or preventing a disease in a subject in need of such treatment or prevention, said method comprising administering to said subject a disease treating or preventing effective amount of a nucleic acid as defined in claim 45, wherein (i) said disease is caused directly or indirectly by an agent, (ii) a symptom of said disease is caused by an agent, or (iii) said disease produces a physical, chemical, or biological response, wherein said agents or response include said target sequence or molecule.

85. A method of treating or preventing a disease in a subject in need of such treatment or prevention, said method comprising administering to said subject a disease treating or preventing effective amount of a pharmaceutical composition as defined in claim 59, wherein (i) said disease is caused directly or indirectly by an agent, (ii) a symptom of said disease is caused by an agent, or (iii) said disease produces a physical, chemical, or biological response, wherein said agents or response include said target sequence or molecule.

86. A method of treating or preventing a disease in a subject in need of such treatment or prevention, said method comprising administering to said subject a disease treating or preventing effective amount of a vaccine as defined in claim 69, wherein (i) said disease is caused directly or indirectly by an agent, (ii) a symptom of said disease is caused by an agent, or (iii) said disease produces a physical, chemical, or biological response, wherein said agents or response include said target sequence or molecule.
